(19) United States
(12) Patent Application Publication
(10) Pub. No.: US 2023/0149278 A1
(43) Pub. Date: May 18, 2023

(54) METHOD FOR DYEING KERATIN MATERIAL, COMPRISING THE USE OF AN ORGANOSILICON COMPOUND, A POLYSACCHARIDE, A COLORING COMPOUND AND A POST-TREATMENT AGENT

(71) Applicant: Henkel AG & Co. KGaA, Duesseldorf (DE)

(72) Inventors: Thomas HIPPE, Appen (DE); Jessica BRENDER, Hamburg (DE); Stefan HOEPFNER, Hamburg (DE)

(73) Assignee: Henkel AG & Co. KGaA, Duesseldorf (DE)

(21) Appl. No.: 17/917,398

(22) PCT Filed: Feb. 11, 2021
(86) PCT No.: PCT/EP2021/053309

§ 371 Date: Oct. 6, 2022

(30) Foreign Application Priority Data
Apr. 8, 2020 (DE) 10 2020 204 541.0

(51) Int. Cl. A61K 8/58; A61K 8/34; A61K 8/81; A61K 8/73; A61Q 5/10

(52) U.S. Cl. CPC A61K 8/585; A61K 8/345; A61K 8/731; A61K 8/8111; A61K 8/8152; A61Q 5/10; A61K 2800/884; A61K 2800/4324

(57) ABSTRACT

The subject of the present disclosure is a process for dyeing keratinous material, in particular human hair, comprising the following steps:
Application of an agent (a) to the keratinous material, wherein the agent (a) comprises:
   (al) at least one organic silicon compound selected from the group of silanes having one, two or three silicon atoms, and
   (a2) an anionic, branched polysaccharide with a repeating tetra saccharide unit,
   (a3) at least one polyol and
   (a4) not more than about 25 wt.% water, and
application of an agent (b) to the keratinous material, wherein the agent (b) comprises:
   (bl) at least one sealing reagent,
wherein at least one of the agents (a) and (b) further comprises at least one colorant compound selected from the group of pigments and/or direct dyes.

Also disclosed are multi-component packaging unit (kit-of-parts) for dyeing keratinous material.

### CROSS-REFERENCE TO RELATED APPLICATION

This application is a U.S. National-Stage entry under 35 U.S.C. § 371 based on International Application No. PCT/EP2021/053309, filed Feb. 11, 2021, which was published under PCT Article 21(2) and which claims priority to German Application No. 102020204541.0, filed Apr. 8, 2020, which are all hereby incorporated in their entirety by reference.

### TECHNICAL FIELD

The subject of the present application is a method for treating keratinous material, in particular human hair, which comprises the application of two agents (a) and (b). The agent (a) is exemplified by its content of at least one organic silicon compound (a1) and a selected polysaccharide (a2). The agent (b) contains at least one sealing reagent (b1). Furthermore, either agent (a) or agent (b) or both agents (a) and (b) contain at least one colorant compound selected from the group of pigments and/or direct dyes.

A further subject of this application is a multi-component packaging unit (kit-of-parts) for dyeing keratinous material, in particular human hair, which comprises separately prepared at least three agents (a′), (a″) and (b). Agents (a′) and (a″) can be used to prepare the agent (a) used in the process described above.

### BACKGROUND

The change in shape and color of keratin fibers, especially hair, is an important area of modem cosmetics. To change the hair color, the expert knows various coloring systems depending on coloring requirements. Oxidation dyes are usually used for permanent, intensive dyeing with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing obtained with direct dyes have a shorter shelf life and quicker wash ability. Dyes with direct dyes usually remain on the hair for a period of between 5 and about 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed without residue by a few washes with surfactant-containing cleaning agents. Various products of this type are available on the market under the name hair mascara.

If the user wants particularly long-lasting dyeing, the use of oxidative dyes has so far been his/her only option. However, despite numerous optimization attempts, an unpleasant ammonia or amine odor cannot be completely avoided in oxidative hair dyeing. The hair damage still associated with the use of oxidative dyes also has a negative effect on the user’s hair.

EP 2168633 B1 deals with the task of producing long-lasting hair colorations using pigments. The publication teaches that when the combination of a pigment, an organic silicon compound, a film-forming polymer and a solvent is used on hair, it is possible to produce colorations that are particularly resistant to abrasion and/or shampooing.

The organic silicon compounds used in the dyes include highly reactive compounds that undergo hydrolysis or oligomerization and/or polymerization in the presence of water and/or in their application. The rate of oligomerization and/or polymerization must be such that it allows the colorant to be applied in a time acceptable to users.

### BRIEF SUMMARY

Methods for dyeing keratin material and a kit-of-parts for the same are provided. A method includes applying an agent (a) to the keratinous material, and applying an agent (b) to the keratinous material. The agent (a) comprises an organic silicon compound (a1) selected from the group of silanes having 1, 2, or 3 silicon atoms, as well as an anionic, branched polysaccharide (a2) with a repeating tetra saccharide unit. The agent (a) also includes a polyol (a3). The agent (a) includes not more than 25 weight percent water, based on a total weight of the agent (a). The agent (b) includes a sealing reagent. At least one of the agents (a) and (b) includes a colorant compound selected from the group of pigments and/or direct dyes.

A kit-of-parts is provided in another embodiments. The kit-of-parts includes a first container containing an agent (a′), a second container containing an agent (a″), and a third container containing an agent (b). The agent (a′) includes an organic silicon compound (a1) selected from the group of silanes having 1, 2, or 3 silicon atoms. The agent (a″) includes an anionic, branched polysaccharide (a2) with a repeating tetra saccharide unit, and a polyol (a3). The agent (a″) includes not more than 25 weight percent water, based on a total weight of the sum of agent (a′) and agent (a″). The agent (b) includes a sealing reagent. At least one of the agents (a″) and (b) further includes a colorant compound selected from the group of pigments and/or direct dyes.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

There is a need to provide hair dyes with pigments that on the one hand have high wash and rub fastness and on the other hand do not negatively affect hair properties such as manageability and feel. For this purpose, it would be desirable that the hair coloring has an optimal comfort of use, especially in terms of application time and application properties of the agent.

Accordingly, the task of the present disclosure was to provide a coloring system with pigments that has fastness properties comparable to oxidative coloring. Wash fastness properties in particular should be outstanding, but the use of oxidation dye precursors normally used for this purpose should be avoided.

Surprisingly, it has now been found that the aforementioned task can be excellently solved if keratinous materials, in particular human hair, are colored by a process in which at least two agents (a) and (b) are applied to the keratinous materials (hair). In this case, the first agent (a) comprises at least one organic silicon compound from the group of silanes having one, two or three silicon atoms (a1) and, furthermore, an anionic, branched polysaccharide having a repeating tetra saccharide unit (a2), at least one polyol (a3) and a maximum of 25 wt.%, based on the total weight of the agent (a), of water (a4). The second agent (b) contains at least one sealing reagent.

When the two agents (a) and (b) were used in a dyeing process, keratinous material could be dyed with particularly high color intensity and high fastness properties.

Furthermore, it has been shown that in addition to the dyeing properties of the entire process, agent (a) could also be optimized with regard to its application properties. It has been shown that the combination of anionic branched polysaccharide with a repeating tetra saccharide unit (a2) and at least one polyol (a3), as well as the limitation of the water content in the agent (a) to a maximum of about 25 wt.%, based on the total weight of the agent (a), results in an oligo and polymerization rate of the organic silicon compound (a1) that is optimal for dyeing keratinous material.

A first object of the present disclosure is a method for coloring keratinous material, in particular human hair, comprising the following steps:
Application of an agent (a) to the keratinous material, wherein the agent (a) comprises:
   (a1) at least one organic silicon compound selected from the group of silanes having one, two or three silicon atoms,
   (a2) an anionic, branched polysaccharide with a repeating tetra saccharide unit,
   (a3) at least one polyol and
   (a4) not more than about 25 wt.% of water, based on the total weight of the agent (a), and
Application of an agent (b) to the keratinous material, wherein the agent (b) comprises:
   (b1) at least one sealing reagent,
wherein at least one of the agents (a) and (b) further comprises at least one colorant compound selected from the group of pigments and/or direct dyes.

In the work leading to the present disclosure, it has been found that the preferential successive application of agents (a) and (b) enables the production of very stable and wash fast colorations on the keratinous materials. Without being limited to this theory, it is believed in this context that the joint application of an organic silicon compound (a1) and an anionic, branched polysaccharide with a repeating tetra saccharide unit (a2) leads to the formation of a particularly resistant film on the keratinous material. Application of the second agent (b) seals the film applied to the keratinous material, making it more resistant to washing and/or abrasion. By incorporating at least one colorant compound selected from the group of pigments and/or direct dyes into at least one of agents (a) and (b), colored films can be obtained.

In this way, the colorant compounds can be permanently fixed to the keratinous material, so that extremely wash fast colorations with good resistance to abrasion and/or shampooing could be obtained.

In particular, by limiting the amount of water in the agent (a) to a maximum of 25 wt. %, based on the total weight of the agent (a), and by adding a polyol (a3), an optimum oligo and polymerization rate of the organic silicon compound (a1) can be achieved.

With the aid of an optimum oligo and polymerization rate of the organic silicon compound (a1), good and uniform staining results are achieved even after short application periods of only a few minutes. This also applies when using the dye on longer keratinous fibers, especially hair. These exposure times of the dyes result in a higher comfort for the user.

By adding an anionic, branched polysaccharide with a repeating tetra saccharide unit, it was not only possible to optimize the application properties of the agent (a), in particular the viscosity of the agent (a), but surprisingly, it was also possible to obtain particularly stable films on the keratinous materials. As a result, extremely rub and wash fast dyeing with good resistance to abrasion and/or shampooing could be obtained.

### Keratinous Material

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair in particular.

### Agents (a) and (b)

In the process described, agents (a) and (b) are applied to the keratinous material, in particular human hair. The two agents (a) and (b) are different from each other.

In other words, a first object of the present disclosure is a method for treating keratinous material, in particular human hair, comprising the following steps:
Application of an agent (a) to the keratinous material, wherein the agent (a) comprises:
   (a1) at least one organic silicon compound selected from the group of silanes having one, two or three silicon atoms, and
   (a2) an anionic, branched polysaccharide with a repeating tetra saccharide unit,
   (a3) at least one polyol and
   (a4) not more than about 25 wt.% of water, based on the total weight of the agent (a), and
Application of an agent (b) to the keratinous material, wherein the agent (b) comprises: (b1) at least one sealing reagent,
wherein at least one of the agents (a) and (b) further comprises at least one colorant compound selected from the group of pigments and/or direct dyes.

### Agent (a)

The agent (a) may be liquid, gel or cream. For the purpose of hair treatment, in particular hair coloring, the agent (a) preferably comprises creams, emulsions, gels or also surfactant-containing foaming solutions, such as shampoos, foam aerosols, foam formulations or other preparations suitable for application to the hair.

The agent (a) may contain water. Preferably, the agent (a) contains at least about 2 wt.% water, based on its total weight. Further preferably, the water content is above about 5 wt.%, still further preferably above about 10 wt.% and particularly preferably above about 15 wt.%, in each case based on the total weight of the agent (a). The agent (a) contains a maximum of about 25 wt.% of water, based on the total weight of the agent (a).

### Organic Silicon Compounds From the Group of Silanes (a1)

As an ingredient (a1) essential to the present disclosure, the agent (a) contains at least one organic silicon compound from the group of silanes having one, two or three silicon atoms.

Particularly preferably, the agent (a) contains at least one organic silicon compound (a1) selected from silanes having one, two or three silicon atoms, the organic silicon compound comprising one or more hydroxyl groups and/or hydrolysable groups per molecule.

These organic silicon compounds (a1) or organic silanes contained in the agent (a) are reactive compounds.

Organic silicon compounds, alternatively called organosilicone compounds, are compounds which either have a direct silicon-carbon bond (Si—C) or in which the carbon is bonded to the silicon atom via an oxygen, nitrogen or sulfur atom. The organic silicon compounds of the present disclosure are compounds containing one to three silicon atoms. Organic silicon compounds preferably contain one or two silicon atoms.

According to IUPAC rules, the term silane chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

In a particularly preferred embodiment, a method is exemplified by the application of an agent (a) to the keratinous material, said agent (a) comprising at least one organic silicon compound (a1) selected from silanes having one, two or three silicon atoms, said organic silicon compound further comprising one or more hydroxyl groups or hydrolysable groups per molecule.

In a very particularly preferred embodiment, a method is exemplified by the application of an agent (a) to the keratinous material, said agent (a) comprising at least one organic silicon compound (a1) selected from silanes having one, two or three silicon atoms, said organic silicon compound further comprising one or more basic chemical functions and one or more hydroxyl groups or hydrolysable groups per molecule.

This basic group or basic chemical function can be, for example, an amino group, an alkylamino group, a dialkylamino group or a trialkylamino group, which is preferably connected to a silicon atom via a linker. Preferably, the basic group is an amino group, a C₁-C₆ alkylamino group or a Di(C₁-C₆) alkylamino group.

The hydrolysable group(s) is (are) preferably a C₁-C₆ alkoxy group, especially an ethoxy group or a methoxy group. It is preferred when the hydrolysable group is directly bonded to the silicon atom. For example, if the hydrolysable group is an ethoxy group, the organic silicon compound preferably contains a structural unit R’R”R”’Si—O—CH2—CH3. The radicals R′, R″ and R‴ represent the three remaining free valences of the silicon atom.

A very particularly preferred method is exemplified in that the agent (a) contains at least one organic silicon compound selected from silanes having one, two or three silicon atoms, the organic silicon compound preferably comprising one or more basic chemical functions and one or more hydroxyl groups or hydrolysable groups per molecule.

Particularly good results were obtained when the agent (a) contains at least one organic silicon (a1) compound of formula (I) and/or (II).

The compounds of formulas (I) and (II) are organic silicon compounds selected from silanes having one, two or three silicon atoms, the organic silicon compound comprising one or more hydroxyl groups and/or hydrolysable groups per molecule.

In another very particularly preferred embodiment, the method is exemplified in that an agent is applied to the keratinous material (or human hair), the agent (a) comprising at least one organic silicon compound (a) of formula (I) and/or (II),

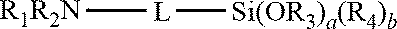

where
R₁, R₂ independently represent a hydrogen atom or a C₁-C₆ alkyl group,
L is a linear or branched bivalent C₁-C₂₀ alkylene group,
R₃ is a hydrogen atom or a C₁-C₆ alkyl group,
R₄ represents a C₁-C₆ alkyl group
a, represents an integer from 1 to 3, and
b stands for the integer 3 a,

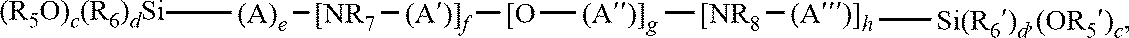

where
R5, R5′, R5″ independently represent a hydrogen atom or a C₁-C₆ alkyl group,
R6, R6′ and R6″ independently represent a C₁-C₆ alkyl group,
A, A′, A″, A‴ and A⁗ independently represent a linear or divalent, bivalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

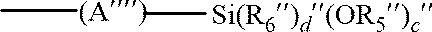

c, stands for an integer from 1 to 3,
d stands for the integer 3 c,
c′ stands for an integer from 1 to 3,
d′ stands for the integer 3 c′,
c“ stands for an integer from 1 to 3,
d“ stands for the integer 3 c“,
e stands for 0 or 1,
f stands for 0 or 1,
g stands for 0 or 1,
h stands for 0 or 1,
provided that at least one of the radicals e, f, g and h is different from 0.

The substituents R₁, R₂, R₃, R₄, R₅, R₅‘, R₅“, R₆, R₆‘, R₆“, R₇, R₈, L, A, A′, A″, A‴ and A⁗ in the compounds of formula (I) and (II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In the organic silicon compounds of the formula (I)

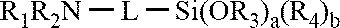

the radicals R₁ and R₂ independently of one another represent a hydrogen atom or a C₁-C₆ alkyl group. Very preferably, R₁ and R₂ both represent a hydrogen atom.

In the middle part of the organic silicon compound is the structural unit or the linker —L— which stands for a linear or branched, divalent C₁-C₂₀ alkylene group.

A divalent C₁-C₂₀ alkylene group may alternatively be referred to as a divalent or divalent C₁-C₂₀ alkylene group, by which is meant that each L grouping may form two bonds. One bond is from the amino group R1R2N to the linker L, and the second bond is between the linker L and the silicon atom.

Preferably, —L— represents a linear, divalent (i.e., divalent) C₁-C₂₀ alkylene group. Further preferably —L— stands for a linear bivalent C₁-C₆ alkylene group. Particularly preferred -L stands for a methylene group (CH₂-), an ethylene group (—CH₂—CH₂—), a propylene group (—CH₂—CH₂—CH₂—) or a butylene (—CH₂—CH₂—CH₂—CH₂—). In particular, L stands for a propylene group (—CH₂—CH₂—CH₂—)

The linear propylene group (—CH₂—CH₂—CH₂—) can alternatively be referred to as the propane-1,3-diyl group.

The organic silicon compounds of formula (I)

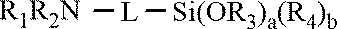

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. In particular, R₃ and R₄ independently of each other represent a methyl group or an ethyl group.

Here a stands for an integer from 1 to 3, and b stands for the integer 3 - a. If a stands for the number 3, then b is equal to 0. If a stands for the number 2, then b is equal to 1. If a stands for the number 1, then b is equal to 2.

Particularly resistant films could be produced if the agent (a) contains at least one organic silicon compound (a1) of formula (I) in which the radicals R₃, R₄ independently of one another represent a methyl group or an ethyl group.

When using the process for dyeing keratinous material, dyeing with the best wash fastness could be obtained analogously when the agent (a) contains at least one organic silicon compound of formula (I) in which the radicals R₃, R₄ independently of one another represent a methyl group or an ethyl group.

Furthermore, dyeing with the best wash fastness could be obtained if the agent (a) contains at least one organic silicon compound of the formula (I) in which the radical a represents the number 3. In this case the radial b stands for the number 0.

In a further preferred embodiment, the agent (a) used in the process is exemplified in that it comprises at least one organic silicon compound (a1) of formula (I), wherein
R₃, R₄ independently of one another represent a methyl group or an ethyl group and
a stands for the number 3 and
b stands for the number 0.

In another preferred embodiment, a method is exemplified in that the agent (a) comprises at least one organic silicon compound (a1) of formula (I),

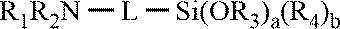

where
— R₁, R₂ both represent a hydrogen atom, and
L represents a linear, bivalent C₁-C₆-alkylene group, preferably a propylene group (—CH₂—CH₂—CH₂—) or an ethylene group (—CH₂—CH₂—),
— R₃ represents a hydrogen atom, an ethyl group or a methyl group,
— R₄ represents a methyl group or an ethyl group,
a stands for the number 3 and
b stands for the number 0.

Organic silicon compounds of the formula (I) which are particularly suitable for solving the problem as contemplated herein are - (3-Aminopropyl)triethoxysilane

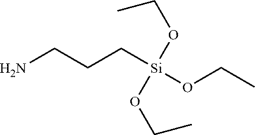

- (3-Aminopropyl)trimethoxysilane

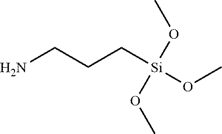

- 1-(3-Aminopropyl)silantriol

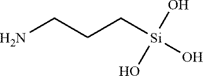

- (2-Aminoethyl)triethoxysilane

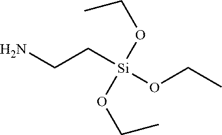

- (2-Aminoethyl)trimethoxysilane

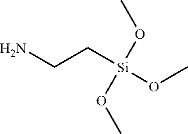

1-(2-Aminoethyl)silantriol

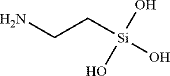

- (3-Dimethylaminopropyl)triethoxysilane

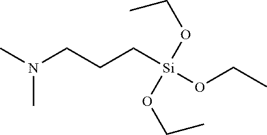 [SMILES: CCO[Si](CCCN(C)C)(OCC)OCC]

- (3-Dimethylaminopropyl)trimethoxysilane

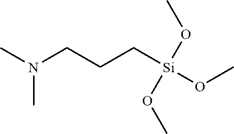

1-(3-Dimethylaminopropyl)silantriol

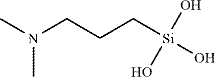

- (2-dimethylaminoethyl)triethoxysilane.

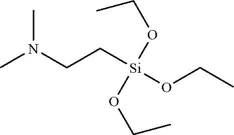

- (2-dimethylaminoethyl)trimethoxysilane and

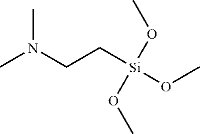

1-(2-Dimethylaminoethyl)silantriol

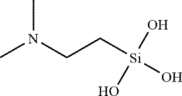

In a further preferred embodiment, a method is exemplified in that the agent (a) comprises at least one organic silicon compound (a1) selected from the group of
(3-Aminopropyl)triethoxysilane
(3-Aminopropyl)trimethoxysilane
1-(3-Aminopropyl)silantriol
(2-Aminoethyl)triethoxysilane
(2-Aminoethyl)trimethoxysilane
1-(2-Aminoethyl)silantriol
(3-Dimethylaminopropyl)triethoxysilane
(3-Dimethylaminopropyl)trimethoxysilane
1-(3-Dimethylaminopropyl)silantriol
(2-Dimethylaminoethyl)triethoxysilane.
(2-Dimethylaminoethyl)trimethoxysilane and/or
1-(2-dimethylaminoethyl)silanetriol.

The aforementioned organic silicon compounds of formula (I) are commercially available. (3-aminopropyl)trimethoxysilane, for example, can be purchased from Sigma-Aldrich®. Also (3-aminopropyl)triethoxysilane is commercially available from Sigma-Aldrich®.

In a further embodiment, the agent comprises at least one organic silicon compound (a1) of formula (II)

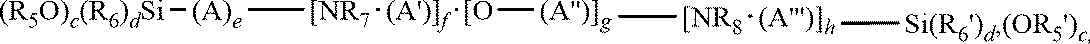

The organosilicon compounds of formula (II) each bear at their two ends the silicon-containing groupings (R₅O)_{c}(R₆)_{d}Si— and —Si(R₆’)_{d’} (OR₅’)_{c}’,

In the central part of the molecule of formula (II) there are the groups —(A)ₑ— and —[NR₇—(A′)]_{f}— and —[O—(A″)]_{g}— and —[NR₈—(A′”)]ₕ-. Here, each of the radicals e, f, g and h can independently of one another stand for the number 0 or 1, with the proviso that at least one of the radicals e, f, g and h is different from 0. In other words, an organic silicon compound of formula (II) contains at least one grouping selected from the group of —(A)— and —[NR₇—(A′)]—and —[O—(A″)]— and —[NR₈—(A″’)]-.

In the two terminal structural units (R₅O)_{c}(R₆)_{d}Si— and — Si(R₆’)_{d’}(OR₅’)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here c stands for an integer from 1 to 3, and d stands for the integer 3 - c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3 - c′. If c′ stands for the number 3, then d′ is 0. If c′ stands for the number 2, then d′ is 1. If c′ stands for the number 1, then d′ is 2.

Films with the highest stability or dyes with the best wash fastness could be obtained when the radicals c and c′ both stand for the number 3. In this case d and d′ both stand for the number 0.

In another preferred embodiment, a method is exemplified in that the agent (a) comprises at least one organic silicon compound (a1) of formula (II),

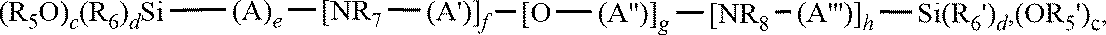

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the present disclosure corresponds to formula (IIa)

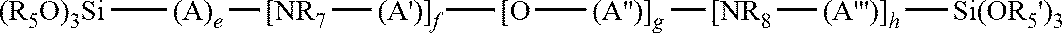

The radicals e, f, g and h can independently stand for the number 0 or 1, whereby at least one radical from e, f, g and h is different from zero. The abbreviations e, f, g and h thus define which of the groupings —(A)ₑ— and —[NR₇—(A′)]_{f}— and —[O—(A″)]_{g}— and —[NR₈—(A′”)]ₕ- are located in the middle part of the organic silicon compound of formula (II).

In this context, the presence of certain groupings has proved to be particularly beneficial in terms of increasing wash ability. Particularly good results were obtained when at least two of the radicals e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

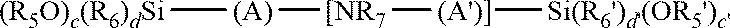

The radicals A, A′, A″, A‴ and A⁗ independently represent a linear or divalent, bivalent C₁-C₂₀ alkylene group. Preferably the radicals A, A′, A″, A‴ and A⁗ independently of one another represent a linear, bivalent C₁-C₂₀ alkylene group. Further preferably the radicals A, A′, A″, A‴ and A⁗ independently represent a linear bivalent C₁-C₆ alkylene group. In particular, the radicals A, A′, A″, A‴ and A⁗ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). Very preferably, the radicals A, A′, A″, A‴ and A⁗ represent a propylene group (—CH₂—CH₂—CH₂—).

The divalent C₁-C₂₀ alkylene group may alternatively be referred to as a divalent or divalent C₁-C₂₀ alkylene group, by which is meant that each grouping A, A′, A″, A‴ and A⁗ may form two bonds.

The linear propylene group (—CH₂—CH₂—CH₂—) can alternatively be referred to as the propane-1,3-diyl group.

If the radical f represents the number 1, then the organic silicon compound of formula (II) contains a structural grouping —[NR₇—(A′)]—. If the radical h represents the number 1, then the organic silicon compound of formula (II) contains a structural grouping —[NR₈—(A′”)]-.

Wherein radicals R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

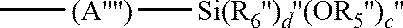

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound contains the grouping [NR₇-(A′)] but not the grouping —[NR₈—(A″’)].. If the radical R7 now stands for a grouping of the formula (III), the agent (a) contains an organic silicon compound with 3 reactive silane groups.

In another preferred embodiment, a method is exemplified in that the agent (a) comprises at least one organic silicon compound (a1) of formula (II),

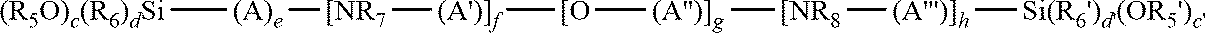

where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a method is exemplified in that the agent (a) comprises at least one organic silicon compound of formula (II), wherein
e and f both stand for the number 1,
g and h both stand for the number 0,
A and A′ independently of one another represent a methylene group(—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (III).

Organic silicon compounds of formula (II) which are well suited for solving the problem as contemplated herein are: - 3-(Trimethoxysilyl)—N—[3-(trimethoxysilyl)propyl]-1-propanamine

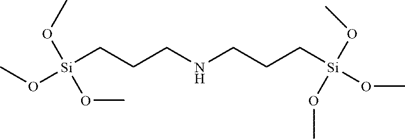

- 3-(Triethoxysilyl)—N—[3-(triethoxysilyl)propyl]-1-propanamine

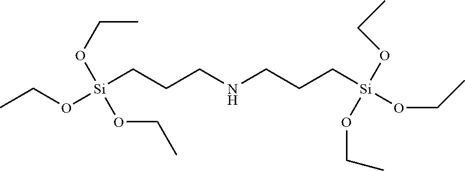 [SMILES: CCO[Si](CCCNCCC[Si](OCC)(OCC)OCC)(OCC)OCC]

- N-Methyl-3-(trimethoxysilyl)-N-[3-(trimethoxysilyl)propyl]-1-propanamine

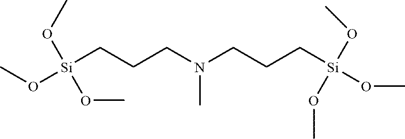

- N-Methyl-3-(triethoxysilyl)-N-[3-(triethoxysilyl)propyl]-1-propanamine

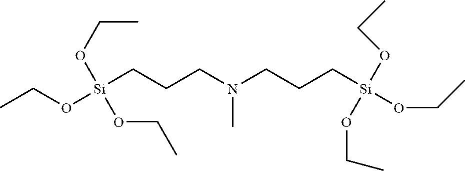 [SMILES: CCO[Si](CCCN(C)CCC[Si](OCC)(OCC)OCC)(OCC)OCC]

- 2-[Bis[3-(trimethoxysilyl)propyl]amino]-ethanol

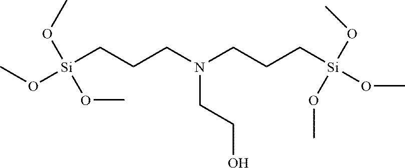

- 2-[Bis[3-(triethoxysilyl)propyl]amino]ethanol

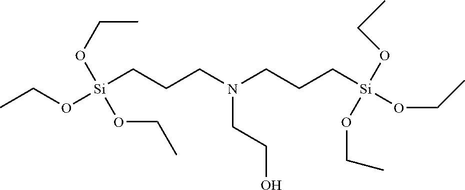 [SMILES: CCO[Si](CCCN(CCO)CCC[Si](OCC)(OCC)OCC)(OCC)OCC]

- 3-(Trimethoxysilyl)-N,N-bis[3-(trimethoxysilyl)propyl]-1-propanamine

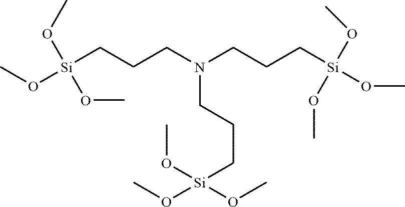

- 3-(Triethoxysilyl)-N,N-bis[3-(triethoxysilyl)propyl]-1-propanamine

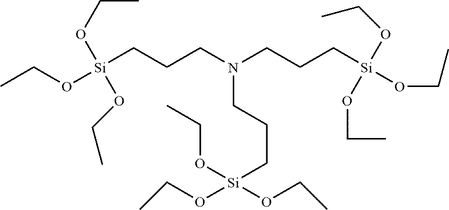 [SMILES: CCO[Si](CCCN(CCC[Si](OCC)(OCC)OCC)CCC[Si](OCC)(OCC)OCC)(OCC)OCC]

- N1,N1-Bis[3-(trimethoxysilyl)propyl]-1,2-ethanediamine,

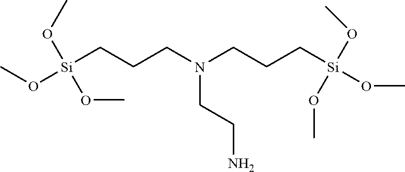

-N1,N1-Bis[3-(triethoxysilyl)propyl]-1,2-ethanediamine,

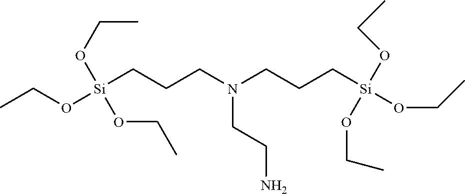 [SMILES: CCO[Si](CCCN(CCN)CCC[Si](OCC)(OCC)OCC)(OCC)OCC]

- N,N-Bis[3-(trimethoxysilyl)propyl]-2-propene-1-amine

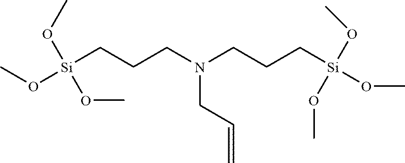

- N,N-Bis[3-(triethoxysilyl)propyl]-2-propene-1-amine

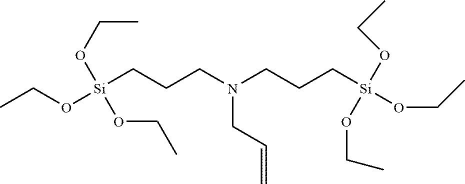 [SMILES: C=CCN(CCC[Si](OCC)(OCC)OCC)CCC[Si](OCC)(OCC)OCC]

The aforementioned organic silicon compounds of formula (II) are commercially available.
Bis(trimethoxysilylpropyl)amines with the CAS number 82985-35-1 can be purchased from Sigma-Aldrich®.
Bis[3-(triethoxysilyl)propyl]amines with the CAS number 13497-18-2 can be purchased from Sigma-Aldrich®, for example. N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich® or Fluorochem®.
3-(triethoxysilyl)-N,N-bis[3-(triethoxysilyl)propyl]-1-propanamine with the CAS number 18784-74-2 can be purchased for example from Fluorochem® or Sigma-Aldrich®.

In a further preferred embodiment, a method is exemplified in that the agent (a) comprises at least one organic silicon compound (a1) selected from the group of
3-(Trimethoxysilyl)-N-[3-(trimethoxysilyl)propyl]-1-propanamine
3-(Triethoxysilyl)-N-[3-(triethoxysilyl)propyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[Bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-Bis[3-(trimethoxysilyl)propyl]-1,2-ethanediamine,
N1,N1-Bis[3-(triethoxysilyl)propyl]-1,2-ethanediamine,
N,N-Bis[3-(trimethoxysilyl)propyl]-2-Propen-1-amine and/or
N,N-Bis[3-(triethoxysilyl)propyl]-2-propen-1-amine.

In further tests, in particular dyeing tests, it has also been found to be particularly advantageous if the agent (a) applied to the keratinous material in the process contains at least one organic silicon compound of the formula (IV)

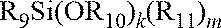

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be called a silane of the alkyl-alkoxy-silane or alkyl-hydroxy-silane type,

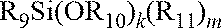

where
R₉ stands for a C₁-C₁₈ alkyl group,
R₁₀ represents a hydrogen atom or a C₁-C₆ alkyl group,
R₁₁ represents a C₁-C₆ alkyl group
k is an integer from 1 to 3, and
m stands for the integer 3 k.

In a further preferred embodiment, the method is exemplified in that the agent (a) comprises at least one organic silicon compound (a1) of formula (IV)

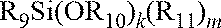

where
R₉ stands for a C₁-C₁₈ alkyl group,
R₁₀ represents a hydrogen atom or a C₁-C₆ alkyl group,
R₁₁ represents a C₁-C₆ alkyl group
k is an integer from 1 to 3, and
m stands for the integer 3 k.

In a further preferred embodiment, a process is exemplified in that the agent (a) comprises, in addition to the organic silicon compound or compounds of formula (I), at least one further organic silicon compound of formula (IV)

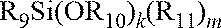

where
R₉ stands for a C₁-C₁₈ alkyl group,
R₁₀ represents a hydrogen atom or a C₁-C₆ alkyl group,
R₁₁ represents a C₁-C₆ alkyl group
k is an integer from 1 to 3, and
m stands for the integer 3 k.

In a further preferred embodiment, a process is exemplified in that the agent (a) comprises, in addition to the organic silicon compound or compounds of formula (II), at least one further organic silicon compound of formula (IV)

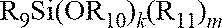

where
R₉ stands for a C₁-C₁₈ alkyl group,
R₁₀ represents a hydrogen atom or a C₁-C₆ alkyl group,
R₁₁ represents a C₁-C₆ alkyl group
k is an integer from 1 to 3, and
m stands for the integer 3 k.

In a further preferred embodiment, a process is exemplified in that the agent (a) comprises, in addition to the organic silicon compound or compounds of formula (I) and/or (II), at least one further organic silicon compound of formula (IV)

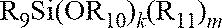

where
R₉ stands for a C₁-C₁₈ alkyl group,
R₁₀ represents a hydrogen atom or a C₁-C₆ alkyl group,
R₁₁ represents a C₁-C₆ alkyl group
k is an integer from 1 to 3, and
m stands for the integer 3 k.

In the organic silicon compounds of formula (IV), the radical R₉ represents a C₁-C₁₈ alkyl group. This C₁-C₁₈ alkyl group is saturated and can be linear or branched. Preferably, R₉ represents a linear C₁-C₁₈ alkyl group. Preferably, R₉ represents a methyl group, an ethyl group, an n-propyl group, an n-butyl group, an n-pentyl group, an n-hexyl group, an n-octyl group, an n-dodecyl group or an n-octadecyl group. Particularly preferably, R₉ represents a methyl group, an ethyl group, an n-hexyl group or an n-octyl group.

In the organic silicon compounds of form (IV), the R₁₀ radical represents a hydrogen atom or a C₁-C₆ alkyl group. Especially preferably, R₁₀ stands for a methyl group or an ethyl group.

In the organic silicon compounds of form (IV), the radical R₁₁ represents a C₁-C₆ alkyl group. Particularly preferably, R₁₁ represents a methyl group or an ethyl group.

Furthermore, k stands for a whole number from 1 to 3, and m stands for the whole number 3 - k. If k stands for the number 3, then m is equal to 0. If k stands for the number 2, then m is equal to 1. If k stands for the number 1, then m is equal to 2.

Particularly stable films, i.e. dyeing with particularly good wash fastness properties, could be obtained if an agent (a) containing at least one organic silicon compound (a1) corresponding to formula (IV): in which the radical k is the number 3, was used in the process. In this case the radical m stands for the number 0.

Organic silicon compounds of the formula (IV) which are particularly suitable for solving the problem as contemplated herein are - Methyltrimethoxysilane

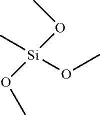

- Methyltriethoxysilane

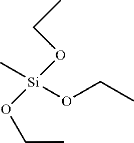

- Ethyltrimethoxysilane

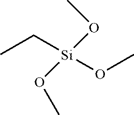

- Ethyltriethoxysilane

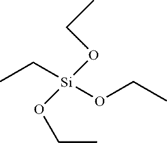

- n-Hexyltrimethoxysilane

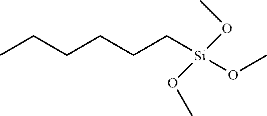

- n-Hexyltriethoxysilane

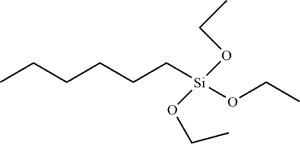 [SMILES: CCCCCC[Si](OCC)(OCC)OCC]

- n-Octyltrimethoxysilane

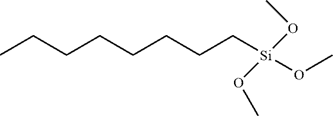

- n-Octyltriethoxysilane

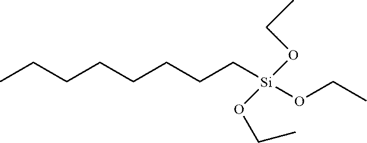 [SMILES: CCCCCCCC[Si](OCC)(OCC)OCC]

- n-dodecyltrimethoxysilane and/or

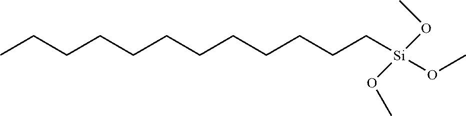 [SMILES: CCCCCCCCCCCC[Si](OC)(OC)OC]

- n-dodecyltriethoxysilane.

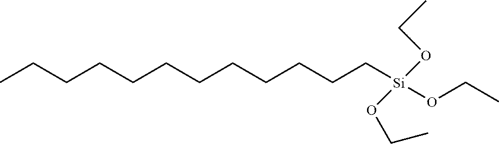 [SMILES: CCCCCCCCCCCC[Si](OCC)(OCC)OCC]

n-octadecyltrimethoxysilane and/or n-octadecyltriethoxysilane.

In another preferred embodiment, a method is exemplified in that the agent (a) comprises at least one organic silicon compound (a1) of formula (IV) selected from the group of
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane.
Octadecyltrimethoxysilane and/or
Octadecyltriethoxysilane.

The organic silicon compounds described above are reactive compounds. In this context, it has been found preferable if the agent (a) contains - based on the total weight of the agent (a) - one or more organic silicon compounds (a1) in a total amount of about 0.1 to about 20 wt. %, preferably about 1 to about 15 wt. % and particularly preferably about 2 to about 8 wt. %.

In a further preferred embodiment, a process is exemplified in that the agent (a) contains - based on the total weight of the agent (a) - one or more organic silicon compounds (a1) in a total amount of about 0.1 to about 20 wt. %, preferably about 1 to about 15 wt. % and particularly preferably about 2 to about 8 wt. %.

To achieve particularly good dyeing results, it is particularly advantageous to use the organic silicon compounds of the formula (I) and/or (II) in certain quantity ranges on agent (a). Particularly preferably, the agent (a) contains - based on the total weight of the agent (a) -one or more organic silicon compounds of the formula (I) and/or (II) in a total amount of about 0.1 to about 10 wt. %, preferably about 0.5 to about 5 wt. % and particularly preferably about 0.5 to about 3 wt. %.

In a further preferred embodiment, a process is exemplified in that the agent (a) contains - based on the total weight of the agent (a) - one or more organic silicon compounds of the formula (I) and/or (II) in a total amount of about 0.1 to about 10 wt. %, preferably about 0.5 to about 5 wt. % and particularly preferably about 0.5 to about 3 wt. %.

Furthermore, it has proven to be particularly preferred if the organic silicon compound(s) of formula (IV) is (are) also present in certain quantity ranges in agent (a). Particularly preferably, the agent (a) contains - based on the total weight of the agent (a) - one or more organic silicon compounds of the formula (IV) in a total amount of about 0.1 to about 20 wt. %, preferably about 2 to about 15 wt. % and particularly preferably about 4 to about 9 wt. %.

In a further preferred embodiment, a process is exemplified in that the agent (a) contains - based on the total weight of the agent (a) - one or more organic silicon compounds of the formula (IV) in a total amount of about 0.1 to about 20 wt. %, preferably about 2 to about 15 wt. % and particularly preferably about 3.2 to about 10 wt. %.

In the course of the work leading to this present disclosure, it was found that particularly stable and uniform films could be obtained on the keratinous material even when the agent (a) contained two organic silicon compounds that were structurally different from each other.

In another preferred embodiment, a method is exemplified in that the agent (a) comprises at least two structurally different organic silicon compounds.

In a preferred embodiment, a process is exemplified in that an agent (a) comprising at least one organic silicon compound of formula (I) and at least one organic silicon compound of formula (IV) is applied to the keratinous material.

In an explicitly very particularly preferred embodiment, a process is exemplified in that there is applied to the keratinous material an agent (a) comprising at least one organic silicon compound of formula (I) selected from the group of (3-aminopropyl)triethoxysilane and (3-aminopropyl)trimethoxysilane and additionally containing at least one organic silicon compound of formula (IV) selected from the group of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In a further preferred embodiment, a method is exemplified in that the agent (a) comprises - based on the total weight of the agent (a):
about 0.5 to about 5 wt. % % of at least one first organic silicon compound (a1) which is selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
about 3.2 to about 10 wt. % of at least one second organic silicon compound (a1) selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

In this embodiment, the agent (a) comprises one or more organic silicon compounds of a first group in a total amount of about 0.5 to about 3 wt. %. The organic silicon compounds of this first group are selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and/or (2-dimethylaminoethyl)triethoxysilane.

In this embodiment, the agent (a) comprises one or more organic silicon compounds of a second group in a total amount of about 3.2 to about 10 wt.%. The organic silicon compounds of this second group are selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in organic silicon compounds with at least one hydrolysable group. The hydrolysis products and/or organic silicon compounds having at least one hydroxy group may react with each other in a condensation reaction. For this reason, both the organosilicon compounds having at least one hydrolysable group and their hydrolysis and/or condensation products may be present in the agent (a). When organosilicon compounds having at least one hydroxyl group are used, both the organic silicon compounds having at least one hydroxyl group and their condensation products may be present in the agent (a).

A condensation product is understood to be a product formed by the reaction of at least two organic silicon compounds each having at least one hydroxyl group or hydrolysable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

Particularly good results were obtained when organic silicon compounds of formula (I) and/or (II) were used in the process. Since, as already described above, hydrolysis/condensation already starts at traces of moisture, the hydrolysis and/or condensation products of the organic silicon compounds (I) and/or (II) are also included in this embodiment.

### Anionic, Branched Polysaccharide With a Repeating Tetra Saccharide Unit (a2)

When agent (a) is applied to the keratinous material, the organic silicon compound(s) (a1), which preferably comprise one or more hydroxyl groups or hydrolysable groups per molecule, are first hydrolyzed and oligomerized or polymerized in the presence of the water. The hydrolysis products or oligomers formed in this way have a particularly high affinity for the surface of the keratinous material and form a film there. If the agent (a) further comprises at least one colorant compound, the film formed on the keratinous material is a colored film. Following the application of agent (a), agent (b) is now applied, whereby the sealing reagent contained in this agent (b) seals the, possibly colored, film. If the agent (b) further contains at least one colorant compound, either the uncolored film produced in the first step is sealed and colored, or the color impression of the colored film produced in the first step is enhanced or modified, depending on the colorant compound used, or the color impression of the first film is enhanced or modified by forming a second, colored film on the first, colored film. If the agent (b) does not contain a colorant compound, the colored film prepared in the first step is sealed. Successive application of agents (a) and (b) produces a coloration that is particularly resistant to external influences.

As an essential ingredient (a2) of the present disclosure, the agent (a) used in the dyeing process contains an anionic branched polysaccharide having a repeating tetra saccharide unit.

It has been shown that the use of an anionic, branched polysaccharide with a repeating tetra saccharide unit leads to particularly stable and intense colorations.

The polysaccharide used is a polysaccharide with tetra saccharide repeating units, which preferably includes D-glucose, L-fucose and D-glucuronic acid in a ratio of about 2:1:1.

In a particularly preferred embodiment, the repeating unit of the polysaccharide has the following sequence of formula I:

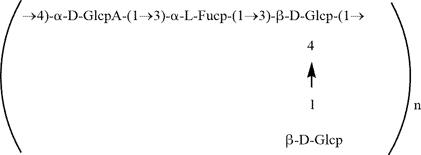

wherein D-GlcpA is a radical of D-glucuronic acid, L-Fucp is a radical of L-fucose, D-Glcp is a radical of D-glucose, where n is an integer between about 2500 and about 3500.

Accordingly, in a preferred embodiment, a process is exemplified in that the agent (a) comprises an anionic branched polysaccharide having a tetra saccharide repeating unit (a2), wherein the tetra saccharide repeating unit includes D-glucose, L-fucose and D-glucuronic acid in a ratio of about 2:1:1.

This anionic polysaccharide preferably has a molecular weight in the order of about 2000 kD.

In general, the polysaccharide can be obtained by the classical techniques of preparation and extraction of polysaccharides (chemical synthesis, enzymatic extraction of exopolysaccharides, etc.). According to an advantageous production method, the polysaccharide is a fully deacetylated exopolysaccharide produced by fermentation of bacteria, in particular Klebsiella aerogenes type 54 strain A3.

A suitable anionic, branched polysaccharide with a repeating tetra saccharide unit is, for example, the polysaccharide with the INCI designation “Biosaccharide Gum-4”, which is available under the name Glycofilm® or PolluStop® from the company Solabia®.

In a preferred embodiment, the process is exemplified in that the agent (a) contains an anionic branched polysaccharide having a repeating tetra saccharide unit (a2) comprising a polysaccharide having the INCI designation “Biosaccharide Gum-4”.

Particularly good results were obtained when the agent (a) contains - based on the total weight of the agent (a) - the anionic, branched polysaccharide with a repeating tetra saccharide unit (a2) in a total amount of from about 0.05 to about 5 wt.%, preferably from about 0.1 to about 3 wt. % and very preferably from about 0.2 to about 2.5 wt.%.

Very particularly good results could be obtained if the agent (a) - based on the total weight of the agent (a) - contains the anionic, branched polysaccharide with a repeating tetra saccharide unit (a2), comprising a polysaccharide with the INCI designation “Biosaccharide Gum-4”, in a total amount of from about 0.05 to about 5 wt.%, preferably from about 0.1 to about 3 wt.% and very particularly preferably from about 0.2 to about 2.5 wt.%.

### Polyol (a3)

As a third ingredient essential to the present disclosure, the agent (a) used in the process comprises at least one polyol (a3).

A polyol is a chemical compound with at least two hydroxyl groups (-OH groups). Suitable polyols may include, in particular, diols and triols.

An aliphatic diol is also known as a glycol. Preferred diols are C₂-C₉ alkanols with two hydroxyl groups and polyethylene glycols with 3 to about 20 ethylene oxide units. The pigment suspension contains at least one C₂-C₉ alkanol with two hydroxyl groups or at least one water-soluble polyethylene glycol with 3 to about 20 ethylene oxide units or mixtures of at least one C₂-C₉ alkanol with two hydroxyl groups and at least one water-soluble polyethylene glycol with 3 to about 20 ethylene oxide units.

Preferably, the C₂-C₉ alkanols with two hydroxyl groups are selected from ethylene glycol, 1,2-propylene glycol, 1,3-propylene glycol, 2-methyl-1,3-propanediol, 1,2-butylene glycol, 1,3-butylene glycol, 1,4-butylene glycol, 1,2-pentanediol, 1, 5-pentanediol, 1,2-hexanediol, 1,6-hexanediol, 1,2-octanediol, 1,8-octanediol, cis-1,4-dimethylolcyclohexane, trans-1,4-dimethylolcyclohexane, any isomeric mixtures of cis- and trans-1,4-dimethylolcyclohexane, and mixtures of these diols. Also suitable diols are diethylene glycol, dipropylene glycol and/or PPG-10 butanediol (INCI). Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20, and mixtures thereof.

Of said diols, the agent (a) used in the process preferably contains at least one diol selected from the group of ethylene glycol, 1,2-propylene glycol, 1,3-propylene glycol, 1,2-butylene glycol, 1,3-butylene glycol, 1,4-butylene glycol, 1,2-pentanediol, 1,5-pentanediol, 1,2-hexanediol, 1,6-hexanediol, PEG-8, PEG-32 and PPG-10 butanediol (INCI).

In a preferred embodiment, the agent (a) used in the process is exemplified in that it contains as polyol at least one diol selected from the group of ethylene glycol, 1,2-propylene glycol and 1,3-propylene glycol, 1,2-propylene glycol being highly preferred.

Suitable triols include, for example, 1,2,4- trihydroxycyclohexane, 1,3,5-trihydroxycyclohexane, glycerol and/or trimethylolpropane. Among the mentioned triplets, the agent (a) used in the process preferably contains glycerol.

It may be preferred that the agent (a) used in the process contains at least two polyols comprising 1,2-propylene glycol and/or glycerol.

Particularly good results were obtained when the agent (a)-based on the total weight of agent (a)-used in the process contains one or more polyols in a total amount of from about 5 to about 80 wt.%, preferably from about 10 to about 75 wt.%, more preferably from about 15 to about 70 wt.% and very particularly preferably from about 20 to about 65 wt.%.

Particularly good results were obtained when the agent (a)-based on the total weight of agent (a)-used in the process contains one or more polyols comprising 1,2-propylene glycol and/or glycerol in a total amount of from about 5 to about 80 wt.%, preferably from about 10 to about 75 wt.%, more preferably from about 15 to about 70 wt.% and very particularly preferably from about 20 to about 65 wt.%.

### pH Value of the Agent (a)

It has been found preferable if the agent (a) is made up in the form of a water-containing agent adjusted to an alkaline pH.

To adjust the pH value, the agent (a) may contain at least one alkalizing agent.

To adjust the desired pH, the agents (a) may therefore also contain at least one alkalizing agent. The pH values for the purposes of the present disclosure are pH values measured at a temperature of 22° C.

As alkalizing agent, agent (a) may contain, for example, ammonia, alkanolamines and/or basic amino acids.

The alkanolamines that can the agent in the compositions are preferably selected from primary amines having a C₂-C₆ alkyl parent carrying at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

Particularly preferred alkanolamines are selected from 2-aminoethan-1-ol and/or 2-amino-2-methylpropan-1-ol. A particularly preferred embodiment is therefore exemplified in that the agent contains, as alkalizing agent, an alkanolamine selected from 2-aminoethan-1-ol and/or 2-amino-2-methylpropan-1-ol.

For the purposes of the present disclosure, an amino acid is an organic compound containing in its structure at least one proton table amino group and at least one —COOH or one —SO₃H group. Preferred amino acids are amino carboxylic acids, especially α-(alpha)-amino carboxylic acids and ω-amino carboxylic acids, whereby α-amino carboxylic acids are particularly preferred.

Basic amino acids are those amino acids which have an isoelectric point pI greater than 7.

Basic α-amino carboxylic acids contain at least one asymmetric carbon atom. In the context of the present disclosure, both possible enantiomers can be used equally as specific compounds or their mixtures, especially as race mates. However, it is particularly advantageous to use the naturally preferred isomeric form, usually in L-configuration.

The basic amino acids are preferably selected from the group formed by arginine, lysine, ornithine and histidine, especially preferably arginine and lysine. In a further particularly preferred embodiment, an agent is therefore exemplified in that the alkalizing agent is a basic amino acid selected from the group of arginine, lysine, ornithine and/or histidine.

In addition, the product may contain other alkalizing agents, especially inorganic alkalizing agents. Inorganic alkalizing agents usable as contemplated herein are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Particularly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-Amino-2-methylpropan-2-ol, 3-aminopropan-1,2-diol, 2-amino-2-methylpropan-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Although the agents (a) are preferably adjusted to pH values in the alkaline range, it may nevertheless be necessary in principle to also use acidifiers in small quantities for fine adjustment of the desired pH value. Acidifiers suitable as contemplated herein are, for example, citric acid, lactic acid, acetic acid or also dilute mineral acids (such as hydrochloric acid, sulfuric acid, phosphoric acid).

However, in the course of the work leading to the present disclosure, it has been found that the presence of the alkalizing agent or the adjustment of the alkaline pH is essential for the formation of resistant films on the keratinous material. The presence of excessive amounts of acids can have a negative effect on the strength of the films. For this reason, it has proved preferable to keep the quantities of acids used in the agent (a) as low as possible. For this reason, it is advantageous if the total amount of organic and/or inorganic acids contained in the agent (a) does not exceed a certain value.

In a further preferred embodiment, a process is exemplified in that the total amount of organic acids from the group comprising citric acid, tartaric acid, malic acid and lactic acid contained in the agent (a) is below about 1 wt. %, preferably below about 0.7 wt. %, more preferably below about 0.5 wt. %, even more preferably below about 0.1 wt. % and most preferably below about 0.01 wt. %.

In a further preferred embodiment, a process is exemplified in that the total amount of inorganic acids from the group comprising hydrochloric acid, sulfuric acid and phosphoric acid contained in the agent (a) is below about 1 wt. %, preferably below about 0.7 wt. %, more preferably below about 0.5 wt. %, still more preferably below about 0.1 wt. % and very particularly preferably below about 0.01 wt. %.

The maximum total amounts of the acids contained in the agent (a) given above are always based on the total weight of the agent (a).

### Agent (b)

The method of treatment of keratinous material includes, in addition to the application of agent (a), the application of agent (b). The agent (b) is exemplified in that it contains at least one sealing reagent (b1).

The agent (b) is a post-treatment agent and the application of agent (b) to the keratinous material treated with agent (a) has the effect of making the colorations obtained in the process more durable. In particular, the use of agent (b) can improve the fastness to washing and the fastness to rubbing of the dyeing obtained in the process.

It is preferred that the sealing reagent comprises a compound selected from the group of film forming polymers, alkalizing agents, acidifying agents, and mixtures thereof.

It may be preferred that the sealing reagent comprises a film-forming polymer.

Polymers are macromolecules with a molecular weight of at least about 1000 g/mol, preferably of at least about 2500 g/mol, particularly preferably of at least about 5000 g/mol, which include identical, repeating organic units. The polymers of the present disclosure may be synthetically produced polymers which are manufactured by polymerization of one type of monomer or by polymerization of different types of monomer which are structurally different from each other. If the polymer is produced by polymerizing a type of monomer, it is called a homo-polymer. If structurally different monomer types are used in polymerization, the resulting polymer is called a copolymer.

The maximum molecular weight of the polymer depends on the degree of polymerization (number of polymerized monomers) and the batch size and is determined by the polymerization method. In terms of the present disclosure, it is preferred if the maximum molecular weight of the film-forming polymer as sealing reagent (b1) is not more than about 10⁷ g/mol, preferably not more than about 10⁶ g/mol, and particularly preferably not more than about 10⁵ g/mol.

As contemplated herein, a film-forming polymer is a polymer which is capable of forming a film on a substrate, for example on a keratinic material or a keratinic fiber. The formation of a film can be demonstrated, for example, by viewing the polymer-treated keratinous material under a microscope.

The film-forming polymers (b1) in the agent (b) can be hydrophilic or hydrophobic.

In a first embodiment, it may be preferred to use at least one hydrophobic film-forming polymer in agent (b).

A hydrophobic polymer is a polymer that has a solubility in water at 25° C. (760 mmHg) of less than 1 wt. %.

The water solubility of the film-forming, hydrophobic polymer can be determined in the following way, for example. 1 g of the polymer is placed in a beaker. Make up to 100 g with water. A stir-bar is added and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1 wt. %.

These include acrylic acid-type polymers, polyurethanes, polyesters, polyamides, polyureas, cellulose polymers, nitrocellulose polymers, silicone polymers, acrylamide-type polymers and polyisoprenes.

Particularly well suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

In a further preferred embodiment, a composition (b) is exemplified in that it comprises at least one film-forming, hydrophobic polymer (b1) selected from the group of the copolymers of acrylic acid, the copolymers of methacrylic acid, the homopolymers or copolymers of acrylic acid esters, the homopolymers or copolymers of methacrylic acid esters homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

The film-forming hydrophobic polymers, which are selected from the group of synthetic polymers, polymers obtainable by radical polymerization or natural polymers, have proved to be particularly suitable for solving the problem as contemplated herein.

Other particularly well-suited film-forming hydrophobic polymers can be selected from the homopolymers or copolymers of olefins, such as cycloolefins, butadiene, isoprene or styrene, vinyl ethers, vinyl amides, the esters or amides of (meth)acrylic acid having at least one C₁-C₂₀ alkyl group, an aryl group or a C₂-C₁₀ hydroxyalkyl group.

Other film-forming hydrophobic polymers may be selected from the homo- or copolymers of isooctyl (meth)acrylate, isononyl (meth)acrylate, 2-ethylhexyl (meth)acrylate, lauryl (meth)acrylate), isopentyl (meth)acrylate, n-butyl (meth)acrylate), isobutyl (meth)acrylate, ethyl (meth)acrylate, methyl (meth)acrylate, tert-butyl (meth)acrylate, stearyl (meth)acrylate, hydroxyethyl (meth)acrylate, 2-hydroxypropyl (meth)acrylate, 3-hydroxypropyl (meth)acrylate and/or mixtures thereof.

Further film-forming hydrophobic polymers can be selected from the homo- or copolymers of (meth)acrylamide, N-alkyl(meth)acrylamides, in particular those with C2-C18 alkyl groups, such as N-ethyl acrylamide, N-tert-butylacrylamide, 1e N-octylacrylamide, N-di(C1-C4) alkyl(meth)acrylamide.

Other preferred anionic copolymers are, for example, copolymers of acrylic acid, methacrylic acid or their C₁-C₆ alkyl esters, as they are marketed under the INCI Declaration Acrylates Copolymers. A suitable commercial product is for example Aculyn® 33 from Rohm & Haas. Copolymers of acrylic acid, methacrylic acid or their C₁-C₆ alkyl esters and the esters of an ethylenically unsaturated acid and an alkoxylated fatty alcohol are also preferred. Suitable ethylenically unsaturated acids are especially acrylic acid, methacrylic acid and itaconic acid; suitable alkoxylated fatty alcohols are especially steareth-20 or ceteth-20.

Very particularly preferred polymers on the market are, for example, Aculyn® 22 (Acrylates/Steareth-20 Methacrylate Copolymer), Aculyn® 28 (Acrylates/Beheneth-25 Methacrylate Copolymer), Structure 2001® (Acrylates/Steareth-20 Itaconate Copolymer), Structure 3001® (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer) or the Rohme und Haas distributed Soltex® OPT (Acrylates/C12-22 Alkyl methacrylate Copolymer).

Suitable polymers based on vinyl monomers may include, for example, the homopolymers and copolymers of N-vinylpyrrolidone, vinylcaprolactam, vinyl-(C1-C6) alkyl-pyrrole, vinyl oxazole, vinyl thiazole, vinyl pyrimidine or vinyl imidazole.

Also particularly suitable are the copolymers octylacrylamide/acrylates/ butylaminoethyl methacrylate copolymer, such as those sold commercially by NATIONAL STARCH under the trade names AMPHOMER® or LOVOCRYL® 47, or the copolymers of acrylates/octylacrylamides sold under the trade names DERMACRYL® LT and DERMACRYL® 79 by NATIONAL STARCH.

Suitable olefin-based polymers include homopolymers and copolymers of ethylene, propylene, butene, isoprene and butadiene.

In another embodiment, the film-forming hydrophobic polymers may be the block copolymers comprising at least one block of styrene or the derivatives of styrene. These block copolymers may be copolymers containing one or more blocks in addition to a styrene block, such as styrene/ethylene, styrene/ethylene/butylene, styrene/butylene, styrene/isoprene, styrene/butadiene. Such polymers are commercially distributed by BASF® under the trade name “Luvitol HSB”.

Surprisingly, it was found that particularly intense and wash fast colorations could be obtained when agent (b) contained at least one film-forming polymer as sealing reagent (b1), which was selected from the group of homopolymers and copolymers of acrylic acid, homopolymers and copolymers of methacrylic acid, homopolymers and copolymers of acrylic acid esters, homopolymers and copolymers of methacrylic acid esters, homopolymers and copolymers of acrylic acid amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

In a further preferred embodiment, a process is exemplified in that the agent (b) comprises at least one film-forming polymer as sealing agent (b1), which is selected from the group of the homopolymers and copolymers of acrylic acid, the homopolymers and copolymers of methacrylic acid, the homopolymers and copolymers of acrylic acid esters, the homopolymers and copolymers of methacrylic acid esters, homopolymers and copolymers of acrylic acid amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

In a further embodiment, it may be preferred to use at least one hydrophilic film-forming polymer as sealing reagent (b1) in agent (b).

A hydrophilic polymer is a polymer that has a solubility in water at 25° C. (760 mmHg) of more than 1 wt. %, preferably more than 2 wt. %.

The water solubility of the film-forming, hydrophilic polymer can be determined in the following way, for example. 1 g of the polymer is placed in a beaker. Make up to 100 g with water. A stir-bar is added and the mixture is heated to 25° C. on a magnetic stirrer while stirring. It is stirred for 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, the solubility of the polymer is more than 1 wt. %.

Nonionic, anionic and cationic polymers can be used as film-forming, hydrophilic polymers.

Suitable film-forming hydrophilic polymers may be selected, for example, from the group comprising polyvinylpyrrolidone (co)polymers, polyvinyl alcohol (co)polymers, vinyl acetate (co)polymers, the carboxyvinyl (co)polymers, the acrylic acid (co)polymers, the methacrylic acid (co)polymers, the natural gums, the polysaccharides and/or the acrylamide (co)polymers.

Furthermore, it is particularly preferred to use polyvinylpyrrolidone (PVP) and/or a vinylpyrrolidone-containing copolymer as film-forming hydrophilic polymer.

In another very particularly preferred embodiment, an agent (b) is exemplified in that it comprises at least one film-forming hydrophilic polymer selected from the group of polyvinylpyrrolidone (PVP) and the copolymers of polyvinylpyrrolidone.

It is further preferred if the agent contains polyvinylpyrrolidone (PVP) as the film-forming hydrophilic polymer. Surprisingly, the wash fastness of the stains obtained with PVP-containing agents (b) was also very good.

Particularly well-suited polyvinylpyrrolidones are available, for example, under the name Luviskol® K from BASF® SE, especially Luviskol® K 90 or Luviskol® K 85 from BASF® SE.

The polymer PVP K30, which is marketed by Ashland (ISP, POI Chemical), can also be used as another explicitly very well suited polyvinylpyrrolidone (PVP). PVP K 30 is a polyvinylpyrrolidone which is highly soluble in cold water and has the CAS number 9003-39-8. The molecular weight of PVP K 30 is about 40000 g/mol.

Other particularly suitable polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115 and available from BASF®.

The use of film-forming hydrophilic polymers (b1) from the group of copolymers of polyvinylpyrrolidone has also led to particularly good and wash fast color results.

Vinylpyrrolidone-vinyl ester copolymers, such as those marketed under the trademark Luviskol® (BASF®), are particularly suitable film-forming hydrophilic polymers. Luviskol® VA 64 and Luviskol® VA 73, both vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are particularly preferred in cosmetic compositions.

Vinylpyrrolidone-vinyl acetate copolymers are marketed under the name Luviskol® VA by BASF® SE. For example, a VP/Vinyl Caprolactam/DMAPA Acrylates copolymer is sold under the trade name Aquaflex® SF-40 by Ashland Inc. For example, a VP/DMAPA acrylates copolymer is marketed by Ashland under the name Styleze® CC-10 and is a highly preferred vinylpyrrolidone-containing copolymer.

Other suitable copolymers of polyvinylpyrrolidone may also be those obtained by reacting N-vinylpyrrolidone with at least one further monomer from the group comprising V-vinylformamide, vinyl acetate, ethylene, propylene, acrylamide, vinylcaprolactam, vinylcaprolactone and/or vinyl alcohol.

In another very particularly preferred embodiment, an agent (b) is exemplified in that it comprises at least one film-forming hydrophilic polymer (b1) selected from the group of polyvinylpyrrolidone (PVP), vinylpyrrolidone/vinyl acetate copolymers, vinylpyrrolidone/styrene copolymers, vinylpyrrolidone/ethylene copolymers, vinylpyrrolidone/propylene copolymers, vinylpyrrolidone/vinylcaprolactam copolymers, vinylpyrrolidone/vinylformamide copolymers and/or vinylpyrrolidone/vinyl alcohol copolymers.

Another suitable copolymer of vinylpyrrolidone is the polymer known under the INCI designation maltodextrin/VP copolymer.

Furthermore, intensively colored keratinous material, especially hair, could be obtained with very good wash fastness properties when a nonionic film-forming hydrophilic polymer was used as the film-forming hydrophilic polymer.

In another embodiment, the agent (b) may comprise at least one nonionic film-forming hydrophilic polymer (b1).

As contemplated herein, a non-ionic polymer is understood to be a polymer which in a protic solvent - such as water - under standard conditions does not carry structural units with permanent cationic or anionic groups, which must be compensated by counter ions while maintaining electron neutrality. Cationic groups include quaternized ammonium groups but not protonated amines. Anionic groups include carboxylic and sulfonic acid groups.

Particular preference is given to products containing, as a non-ionic, film-forming, hydrophilic polymer, at least one polymer selected from the group of
Polyvinylpyrrolidone,
Copolymers of N-vinylpyrrolidone and vinyl esters of carboxylic acids containing 2 to 18 carbon atoms, in particular of N-vinylpyrrolidone and vinyl acetate,
Copolymers of N-vinylpyrrolidone and N-vinylimidazole and methacrylamide,
Copolymers of N-vinylpyrrolidone and N-vinylimidazole and acrylamide,
Copolymers of N-vinylpyrrolidone with N,N-di(C1 to C4)alkylamino-(C2 to C4)alkyl acrylamide.

If copolymers of N-vinylpyrrolidone and vinyl acetate are used, it is again preferable if the molar ratio of the structural units contained in the monomer N-vinylpyrrolidone to the structural units of the polymer contained in the monomer vinyl acetate is in the range from about 20:80 to about 80:20, in particular from about 30:70 to about 60:40. Suitable copolymers of vinyl pyrrolidone and vinyl acetate are available, for example, under the trademarks Luviskol® VA 37, Luviskol® VA 55, Luviskol® VA 64 and Luviskol® VA 73 from BASF® SE.

Another particularly preferred polymer is selected from the INCI designation VP/Methacrylamide/Vinyl Imidazole Copolymer, which is available under the trade name Luviset® Clear from BASF® SE.

Another very particularly preferred nonionic, film-forming, hydrophilic polymer is a copolymer of N-vinylpyrrolidone and N,N-dimethylaminopropylmethacrylamide, which is sold, for example, by the company ISP under the INCI designation VP/DMAPA Acrylates Copolymer, e.g. under the trade name Styleze® CC 10.

A cationic polymer is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI designation: Polyquatemium-69), which is marketed, for example, under the trade name AquaStyle® 300 (28-32 wt.% active substance in ethanol-water mixture, molecular weight 350000) by ISP.

Other suitable film-forming, hydrophilic polymers include
Vinylpyrrolidone-vinylimidazolium methochloride copolymers, as offered under the designations Luviquat® FC 370, FC 550 and the INCI designation Polyquaternium-16 as well as FC 905 and HM 552,
Vinylpyrrolidone-vinylcaprolactam-acrylate terpolymers, as they are commercially available with acrylic acid esters and acrylic acid amides as a third monomer component, for example under the name Aquaflex® SF 40.

Polyquatemium-11 is the reaction product of diethyl sulfate with a copolymer of vinyl pyrrolidone and dimethylaminoethyl methacrylate. Suitable commercial products are available under the names Dehyquart® CC 11 and Luviquat® PQ 11 PN from BASF® SE or Gafquat® 440, Gafquat® 734, Gafquat® 755 or Gafquat® 755N from Ashland Inc..

Polyquaternium-46 is the reaction product of vinylcaprolactam and vinylpyrrolidone with methylvinylimidazolium methosulfate and is available for example under the name Luviquat® Hold from BASF® SE. Polyquatemium-46 is preferably used in an amount of 1 to 5 wt. % - based on the total weight of the cosmetic composition. It particularly prefers to use polyquaternium-46 in combination with a cationic guar compound. It is even highly preferred that polyquaternium-46 is used in combination with a cationic guar compound and polyquaternium-11.

Suitable anionic film-forming, hydrophilic polymers can be, for example, acrylic acid polymers, which can be in non-crosslinked or crosslinked form. Such products are sold commercially under the trade names Carbopol® 980, 981, 954, 2984 and 5984 by Lubrizol® or under the names Synthalen® M and Synthalen® K by 3V Sigma® (The Sun Chemicals, Inter Harz).

Examples of suitable film-forming, hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Suitable film-forming, hydrophilic polymers from the acrylamide group are, for example, polymers prepared from monomers of (meth)acrylamido-C1-C4-alkyl sulfonic acid or salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Preferred polymers of poly(meth)arylamido-C1-C4-alkyl-sulfonic acids are crosslinked and at least about 90% neutralized. These polymers can be crosslinked or non-crosslinked.

Cross-linked and fully or partially neutralized polymers of the poly-2-acrylamido-2-methylpropane sulfonic acid type are known under the INCI designation “Ammonium Polyacrylamido-2-methyl-propanesulphonate” or “Ammonium Polyacryldimethyltauramide”.

Another preferred polymer of this type is the crosslinked poly-2-acrylamido-2methyl-propanesulfonic acid polymer sold by Clariant® under the trade name Hostacerin® AMPS, which is partially neutralized with ammonia.

In another explicitly very particularly preferred embodiment, a process is exemplified in that the agent (b) comprises at least one anionic, film-forming, polymer (b1).

In this context, the best results were obtained when the agent (b) contains, as sealing reagent (b1), at least one film-forming polymer comprising at least one structural unit of formula (P-I) and at least one structural unit of formula (P-II)

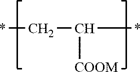

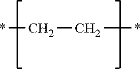

where M is a hydrogen atom or ammonium (NH₄), sodium, potassium, ½ magnesium or ½ calcium.

In a further preferred embodiment, a process is exemplified in that the agent (b) comprises at least one film-forming polymer as sealing reagent (b1), which comprises at least one structural unit of the formula (P-I) and at least one structural unit of the formula (P-II)

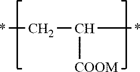

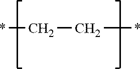

where M is a hydrogen atom or ammonium (NH₄), sodium, potassium, ½ magnesium or ½ calcium.

When M represents a hydrogen atom, the structural unit of the formula (P-I) is based on an acrylic acid unit.

When M stands for an ammonium counter ion, the structural unit of the formula (P-I) is based on the ammonium salt of acrylic acid.

When M stands for a sodium counter ion, the structural unit of the formula (P-I) is based on the sodium salt of acrylic acid.

When M stands for a potassium counter ion, the structural unit of the formula (P-I) is based on the potassium salt of acrylic acid.

If M stands for a half equivalent of a magnesium counter ion, the structural unit of the formula (P-I) is based on the magnesium salt of acrylic acid. If M stands for a half equivalent of a calcium counter ion, the structural unit of the formula (PI) is based on the calcium salt of acrylic acid.

The film-forming polymer or polymers (b1) are preferably used in certain ranges of amounts in the agent (b). In this context, it has proved particularly preferable for solving the problem as contemplated herein if the agent (b) contains - based on the total weight of the agent (b) - one or more film-forming polymers (b1) in a total amount of from about 0.1 to about 18 wt.%, preferably from about 1 to about 16 wt.%, more preferably from about 5 to about 14.5 wt.% and very particularly preferably from about 8 to about 12 wt.%.

In a further preferred embodiment, a process is exemplified in that the agent (b) contains - based on the total weight of the agent (b) - one or more film-forming polymers (b1) in a total amount of from about 0.1 to about 18 wt.%, preferably from about 1 to about 16 wt.%, more preferably from about 5 to about 14.5 wt.% and very particularly preferably from about 8 to about 12 wt.%.

The application of agent (b) comprising a film-forming polymer as sealing reagent (b1) is intended to seal and/or fix the colored film initially produced by the application of agent (a). With application of the second agent (b) with a film-forming polymer as sealing reagent (b1), the film-forming polymer (b1) is deposited on the colored film produced in the first layer in the form of a further film. The multilayer film system created in this way exhibits improved resistance to external influences.

Here, the film produced by the agent (b) comprising a film-forming polymer as sealing reagent (b1) is preferably not colored itself. In this way, it can also be ensured that any abrasion to a certain extent of the second film formed by agent (b) does not lead to any color changes in the entire film system. It is therefore particularly preferred if the agent (b) contains no or only very small amounts of colorant compounds.

In an alternative embodiment, the sealing reagent (b1) contains an alkalizing agent.

Particularly preferably, the alkalizing agent is selected from the group of ammonia, C₂-C₆ alkanolamines, basic amino acids, alkali metal hydroxides and alkaline earth metal hydroxides.

In another particularly preferred embodiment, a process is exemplified in that the agent (b) contains at least one alkalizing agent as sealing reagent (b1), which is selected from the group of ammonia, C₂-C₆ alkanolamines, basic amino acids, alkali metal hydroxides, alkaline earth metal hydroxides, alkali metal silicates, alkali metal metasilicates, alkaline earth metal silicates, alkaline earth metal metasilicates, alkali metal carbonates and alkaline earth metal carbonates.

It has been found that after-treatment with an agent (b) containing ammonia exerts a particularly good influence on improving the wash fastness and rub fastness of the dyeing obtained in the process.

In the context of a further very particularly preferred embodiment, a method is exemplified in that the agent (b) comprises ammonia as sealing reagent (b1).

Good results were also obtained when agent (b) contained at least one C₂-C₆ alkanolamine as sealing reagent (b1).

The alkanolamines that can be used in agent (b) can be selected, for example, from the group of primary amines having a C₂-C₆ alkyl parent carrying at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

In a further preferred embodiment, a process as contemplated herein is exemplified in that the agent (b) comprises, as sealing reagent (b1), at least one alkalizing agent from the group of alkanolamines, which is preferably selected from the group of 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol and 2-amino-2-methylpropane-1,3-diol.

Likewise, good results were obtained when agent (b) contained at least one basic amino acid as sealing reagent (b1).

For the purposes of the present disclosure, an amino acid is an organic compound containing in its structure at least one proton table amino group and at least one —COOH or one —SO₃H group. Preferred amino acids are amino carboxylic acids, especially α-(alpha)-amino carboxylic acids and ω-amino carboxylic acids, whereby α-amino carboxylic acids are particularly preferred.

As contemplated herein, basic amino acids are those amino acids which have an isoelectric point pI of greater than 7.0.

Basic α-amino carboxylic acids contain at least one asymmetric carbon atom. In the context of the present disclosure, both possible enantiomers can be used equally as specific compounds or their mixtures, especially as race mates. However, it is particularly advantageous to use the naturally preferred isomeric form, usually in L-configuration.

The basic amino acids are preferably selected from the group formed by arginine, lysine, ornithine and histidine, especially preferably arginine and lysine. In a further particularly preferred embodiment, the method is therefore exemplified in that the sealing reagent (b1) is an alkalizing agent comprising a basic amino acid selected from the group of arginine, lysine, ornithine and/or histidine.

In a further preferred embodiment, the method is exemplified in that the agent (b) comprises as sealing reagent (b1) at least one alkalizing agent selected from the group of basic amino acids, which is preferably selected from the group of arginine, lysine, ornithine and histidine.

Good results were also obtained when the agent (b) contained at least one alkali metal hydroxide as sealing reagent (b1).Examples of well-suited alkali metal hydroxides are sodium hydroxide and potassium hydroxide.

Good results were also obtained when the agent (b) contained, as sealing reagent (b1), an alkalizing agent comprising at least one alkaline earth metal hydroxide. Suitable alkaline earth metal hydroxides include magnesium hydroxide, calcium hydroxide and barium hydroxide.

Good results were also obtained when the agent (b) contained at least one alkali metal silicate and/or alkali metal metasilicate as sealing reagent (b1).Suitable alkali metal silicates include sodium silicate and potassium silicate. Suitable alkali metal metasilicates include sodium metasilicate and potassium metasilicate.

Good results were also obtained when the agent (b) contained at least one alkali metal carbonate and/or alkaline earth metal carbonate as sealing reagent (b1).Suitable alkali metal carbonates include sodium carbonate and potassium carbonate. Suitable alkaline earth metal carbonates include magnesium carbonate and calcium carbonate.

Within the group of the aforementioned sealing reagents (b1) in the form of an alkalizing agent, ammonia, C₂-C₆ alkanolaminenes, basic amino acids and alkali metal hydroxides have proved to be particularly suitable.

In the context of a further particularly preferred embodiment, the process is exemplified in that the agent (b) comprises as sealing reagent (b1) at least one alkalizing agent selected from the group of ammonia, C₂-C₆ alkanolamines, basic amino acids and alkali metal hydroxides.

In another particularly preferred embodiment, the process is exemplified in that the agent (b) comprises, as sealing reagent (b1), at least one alkalizing agent selected from the group of ammonia, 2-aminoethan-1-ol, 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol, arginine, lysine, omithine, histidine, sodium hydroxide and potassium hydroxide.

The agent (b) contains the alkalizing agent as a sealing reagent (b1) in a cosmetic carrier, preferably in an aqueous cosmetic carrier.

In this context, it has been found preferable if the agent (b) contains - based on the total weight of the agent (b) - about 5.0 to about 99.0 wt. %, preferably about 15.0 to about 97.0 wt. %, more preferably about 25.0 to about 97.0 wt. %, still more preferably about 35.0 to about 97.0 wt. % and very particularly preferably about 45.0 to about 97.0 wt. % of water.

In the context of a further embodiment, the process is exemplified in that the agent (b) contains - based on the total weight of the agent (b) - about 5.0 to about 99.0 wt. %, preferably about 15.0 to about 97.0 wt. %, more preferably about 25.0 to about 97.0 wt. %, still more preferably about 35.0 to about 97.0 wt. % and very particularly preferably about 45.0 to about 97.0 wt. % of water.

The alkalizing agents contained in the agent (b) exert an influence on the pH value of the agent (b). It was found that certain alkaline pH values in particular have a beneficial effect on the dyeing performance achievable in the process and the fastness properties of the dyeing.

For this reason, it is preferred that the agent (b) comprising an alkalizing agent as sealing reagent (b1) has a pH of from about 7.0 to about 12.0, preferably from about 7.5 to about 11.5, more preferably from about 8.0 to about 11.0, and most preferably from about 8.5 to about 9.5.

The pH value can be measured using the usual methods known from the state of the art, such as pH measurement using glass electrodes via combination electrodes or using pH indicator paper.

In another very particularly preferred embodiment, the process is exemplified in that the agent (b) contains an alkalizing agent as sealing reagent (b1) and has a pH of from about 7.0 to about 12.0, preferably from about 7.5 to about 11.5, more preferably from about 8.0 to about 11.0 and most preferably from about 8.5 to about 9.5.

The pH values for the purposes of the present disclosure are pH values measured at a temperature of 22° C.

In a still further alternative embodiment, the sealing reagent (b1) contains an acidifying agent.

Particularly preferably, the acidifying agent is selected from the group of inorganic acids, organic acids and mixtures thereof.

Good results could be obtained when agent (b) contains at least one inorganic acid as sealing reagent (b1).Suitable inorganic acids are, for example, phosphoric acid, sulfuric acid and/or hydrochloric acid, with sulfuric acid being particularly preferred.

In a further preferred embodiment, the process is exemplified in that the agent (b) comprises, as sealing reagent (b1), at least one acidifying agent selected from the group of inorganic acids, which is preferably selected from the group of phosphoric acid, sulfuric acid, hydrochloric acid and mixtures thereof.

In a further, even more preferred embodiment, the method is exemplified in that the agent (b) contains sulfuric acid as sealing reagent (b1).

Good results were also obtained when agent (b) contained at least one organic acid as sealing reagent (b1).The organic acid is preferably selected from the group of formic acid, acetic acid, propionic acid, butyric acid, isobutyric acid, valeric acid, isovaleric acid, pivalic acid, oxalic acid, malonic acid, succinic acid, glutaric acid, glyceric acid, Glyoxylic acid, adipic acid, pimelic acid, corkic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoylic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6, 7-napthalenepentaacetic acid, malonaldehyde acid, 4-hydroxy-phthalamic acid, 1-pyrazolecarboxylic acid, gallic acid or propane tricarboxylic acid, glycolic acid, gluconic acid, lactic acid, maleic acid, ascorbic acid, malic acid, tartaric acid, citric acid and mixtures thereof.

In a further preferred embodiment, the method is exemplified in that the agent (b) comprises as sealing reagent (b1) at least one acidifying agent selected from the group of organic acids, wherein the organic acid is preferably selected from the group of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, corkic acid, azelaic acid, sebacic acid, propiolic acid, crotonic acid, isocrotonic acid, elaidic acid, Maleic acid, fumaric acid, muconic acid, citraconic acid, mesaconic acid, camphoric acid, benzoic acid, o,m,p-phthalic acid, naphthoic acid, toluoylic acid, hydratropasic acid, atropasic acid, cinnamic acid, isonicotinic acid, nicotinic acid, bicarbamic acid, 4,4′-dicyano-6,6′-binicotinic acid, 8-carbamoyloctanoic acid, 1,2, 4-pentane tricarboxylic acid, 2-pyrrole carboxylic acid, 1,2,4,6,7-napthalene pentaacetic acid, malonaldehyde acid, 4-hydroxy-phthalamic acid, 1-pyrazole carboxylic acid, gallic acid or propane tricarboxylic acid, glycolic acid, gluconic acid, lactic acid, maleic acid, ascorbic acid, malic acid, tartaric acid, citric acid and mixtures thereof.

In a further, even more preferred embodiment, the method is exemplified in that the agent (b) contains acetic acid as sealing reagent (b1).

Also suitable acidifiers include methanesulfonic acid and/or 1-hydroxyethane-1,1-diphosphonic acid.

Within the group of the above-mentioned sealing reagents (b1) in the form of an acidifying agent, sulfuric acid and/or acetic acid have proved to be particularly suitable.

In the context of a further particularly preferred embodiment, the process is exemplified in that the agent (b) comprises as sealing reagent (b1) at least one acidifying agent selected from the group of sulfuric acid, acetic acid and mixtures thereof. The agent (b) contains the acidifying agent as sealing reagent (b1) in a cosmetic carrier, preferably in an aqueous cosmetic carrier.

The acidifying agents contained in the agent (b) exert an influence on the pH of the agent (b). It was found that acidic pH values also have a beneficial effect on the dyeing performance achievable in the process and the fastness properties of the dyeing.

For this reason, it is preferred that the agent (b) comprising an acidifying agent as sealing reagent (b1) has a pH of from about 2.0 to about 6.5, preferably from about 3.0 to about 6.0, more preferably from about 4.0 to about 6.0, and most preferably from about 4.5 to about 5.5.

The pH value can be measured using the usual methods known from the state of the art, such as pH measurement using glass electrodes via combination electrodes or using pH indicator paper.

In another very particularly preferred embodiment, the process is exemplified in that the agent (b) contains an acidifying agent as sealing reagent (b1) and has a pH of from about 2.0 to about 6.5, preferably from about 3.0 to about 6.0, more preferably from about 4.0 to about 6.0, and most preferably from about 4.5 to about 5.5.

The pH values for the purposes of the present disclosure are pH values measured at a temperature of 22° C.

### Other Ingredients in Agents (a) and (b)

The agents (a) and (b) described above may also contain one or more optional ingredients. However, it is essential to the present disclosure that at least one of the agents (a) and (b) further comprises at least one colorant compound selected from the group of pigments and/or direct dyes.

It may be preferred that, in addition to the at least one organic silicon compound selected from the group of silanes having one, two or three silicon atoms (a1), the anionic branched polysaccharide having a repeating tetra saccharide unit (a2), the at least one polyol and the water (a4), the agent (a) further comprises at least one coloring compound selected from the group of pigments and/or direct dyes.

Alternatively, it may be preferred that the agent (b) further comprises, in addition to the sealing reagent (b1), at least one colorant compound selected from the group of pigments and/or direct dyes. In an equally preferred embodiment of the process, the agent (a) and the agent (b) each further comprise at least one colorant compound selected from the group of pigments and/or direct dyes.

Irrespective of agent (a) and/or (b), the use of pigments has proved to be particularly preferred in this context.

In another very particularly preferred embodiment, a process is exemplified in that the agent (a) and/or the agent (b) further comprises at least one color-imparting compound selected from the group of pigments.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A beaker glass is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

Suitable pigments can be of inorganic and/or organic origin.

In a preferred embodiment, a process is exemplified in that wherein the agent (a) and/or the agent (b) further comprises at least one colorant compound selected from the group of inorganic and/or organic pigments.

Preferred pigments are selected from synthetic or natural inorganic pigments. Inorganic pigments of natural origin can be produced, for example, from chalk, ochre, umber, green earth, fired Terra di Siena or graphite. Furthermore, black pigments such as iron oxide black, colored pigments such as ultramarine or iron oxide red, and fluorescent or phosphorescent pigments can be used as inorganic pigments.

Particularly suitable are colored metal oxides, hydroxides and oxide hydrates, mixed-phase pigments, sulfur-containing silicates, silicates, metal sulfides, complex metal cyanides, metal sulfates, chromates and/or molybdates. Particularly preferred pigments are black iron oxide (CI 77499), yellow iron oxide (CI 77492), red and brown iron oxide (CI 77491), manganese violet (CI 77742), ultramarines (sodium aluminum sulfosilicates, CI 77007, Pigment Blue 29), chromium oxide hydrate (CI77289), iron blue (ferric ferrocyanide, CI77510) and/or carmine (cochineal).

Also particularly preferred pigments are colored pearlescent pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

Accordingly, a preferred process is exemplified in that the agent (a) and/or the agent (b) further comprises at least one colorant compound selected from the group of pigments selected from the group of colored metal oxides, metal hydroxides, metal oxide hydrates, silicates, metal sulfides, complex metal cyanides, metal sulfates, bronze pigments and/or colored pigments based on natural or synthetic mica coated with at least one metal oxide and/or a metal oxychloride.

A preferred suitable pigment based on synthetic mica is, for example, Timiron® SynWhite Satin from Merck®.

In a further preferred embodiment, the process is exemplified in that the agent (a) and/or the agent (b) comprises at least one colorant compound from the group of pigments selected from pigments based on natural or synthetic mica which are reacted with one or more metal oxides from the group comprising titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

In yet another preferred embodiment of the method, agent (a) and/or agent (b) is exemplified in that it comprises at least one color-imparting compound selected from the group of inorganic pigments, black iron oxide (CI 77499), yellow iron oxide (CI 77492), red iron oxide (CI 77491) and mixtures thereof.

Yellow iron oxide (or iron oxide yellow) is the name for FeO(OH), in the color index under C.I. Pigment Yellow 42 listed.

Red iron oxide (or iron oxide red) is the name for Fe₂O₃, in the color index under C.I. Pigment Red 101 listed. Depending on the particle size, red iron oxide pigments can be adjusted to be very yellowish (small particle size) to very blueish (coarse particles).

Black iron oxide (or iron oxide black) is listed in the Color Index under C.I. Pigment Black 11 listed. Iron oxide black is ferromagnetic. The chemical formula is often given as Fe₃O₄, in reality there is a solid solution of Fe₂O₃ and FeO with inverse spinel structure. Further black pigments are obtained by doping with chromium, copper or manganese.

Brown Black Iron Oxide (or Iron Oxide Brown) usually does not refer to a defined pigment, but to a mixture of yellow, red and/or black iron oxide.

Iron oxide pigments usually have particle diameters in the range of about 2,000 to about 4,000 nm. For some applications, especially for cosmetic purposes, it may be advantageous to use iron oxide pigments with significantly smaller particle diameters. For example, hair dyes with iron oxide pigments that have a particle diameter in the range of about 100 to about 1,000 nm, more preferably about 150 nm to about 700 nm, show better durability and better gray coverage.

Accordingly, a process is preferred in which agent (a) and/or agent (b) further comprises a colorant compound selected from the group of pigments and/or direct dyes, wherein the colorant compound comprises a pigment selected from the group of iron oxide pigments.

Even more preferred is a process wherein agent (a) and/or agent (b) further comprises a colorant compound selected from the group of pigments and/or direct dyes, wherein the colorant compound comprises a pigment selected from the group of iron oxide pigments, and wherein the iron oxide pigment has a particle diameter in the range of about 100 to about 1,000 nm, more preferably about 150 nm to about 700 nm.

Other suitable pigments are based on metal oxide-coated platelet-shaped borosilicates. These are coated with tin oxide, iron oxide(s), silicon dioxide and/or titanium dioxide, for example. Such borosilicate-based pigments are available, for example, under the name MIRAGE® from Eckart® or Reflecks from BASF® SE.

Examples of particularly suitable pigments are commercially available under the trade names Rona®, Colorona®, Xirona®, Dichrona® and Timiron® from Merck®, Ariabel® and Unipure® from Sensient®, Prestige® or SynCrystal® from Eckart® Cosmetic Colors, Flamenco®, Cellini®, Cloisonné®, Duocrome®, Gemtone®, Timica®, MultiReflections, Chione from BASF® SE and Sunshine® from Sunstar®.

Very particularly preferred pigments with the trade name Colorona® are, for example:
Colorona® Copper, Merck®, MICA, CI 77491 (IRON OXIDES)
Colorona® Copper Fine, Merck®, MICA, CI 77491 (IRON OXIDES)
Colorona® Passion Orange, Merck®, Mica, CI 77491 (Iron Oxides), Alumina
Colorona® Patina Silver, Merck®, MICA, CI 77499 (IRON OXIDES), CI 77891 (TITANIUM DIOXIDE)
Colorona® RY, Merck®, CI 77891 (TITANIUM DIOXIDE), MICA, CI 75470 (CARMINE)
Colorona® Oriental Beige, Merck®, MICA, CI 77891 (TITANIUM DIOXIDE), CI 77491 (IRON OXIDES)
Colorona® Dark Blue, Merck®, MICA, TITANIUM DIOXIDE, FERRIC FERROCYANIDE
Colorona® Chameleon, Merck®, CI 77491 (IRON OXIDES), MICA
Colorona® Aborigine Amber, Merck®, MICA, CI 77499 (IRON OXIDES), CI 77891 (TITANIUM DIOXIDE)
Colorona® Blackstar Blue, Merck®, CI 77499 (IRON OXIDES), MICA
Colorona® Patagonian Purple, Merck®, MICA, CI 77491 (IRON OXIDES), CI 77891 (TITANIUM DIOXIDE), CI 77510 (FERRIC FERROCYANIDE)
Colorona® Red Brown, Merck®, MICA, CI 77491 (IRON OXIDES), CI 77891 (TITANIUM DIOXIDE)
Colorona® Russet, Merck®, CI 77491 (TITANIUM DIOXIDE), MICA, CI 77891 (IRON OXIDES)
Colorona® Imperial Red, Merck®, MICA, TITANIUM DIOXIDE (CI 77891), D&C RED NO. 30 (CI 73360)
Colorona® Majestic Green, Merck®, CI 77891 (TITANIUM DIOXIDE), MICA, CI 77288 (CHROMIUM OXIDE GREENS)
Colorona® Light Blue, Merck®, MICA, TITANIUM DIOXIDE (CI 77891), FERRIC FERROCYANIDE (CI 77510)
Colorona® Red Gold, Merck®, MICA, CI 77891 (TITANIUM DIOXIDE), CI 77491 (IRON OXIDES)
Colorona® Gold Plus MP 25, Merck®, MICA, TITANIUM DIOXIDE (CI 77891), IRON OXIDES (CI 77491)
Colorona® Carmine Red, Merck®, MICA, TITANIUM DIOXIDE, CARMINE
Colorona® Blackstar Green, Merck®, MICA, CI 77499 (IRON OXIDES)
Colorona® Bordeaux, Merck®, MICA, CI 77491 (IRON OXIDES)
Colorona® Bronze, Merck®, MICA, CI 77491 (IRON OXIDES)
Colorona® Bronze Fine, Merck®, MICA, CI 77491 (IRON OXIDES)
Colorona® Fine Gold MP 20, Merck®, MICA, CI 77891 (TITANIUM DIOXIDE), CI 77491 (IRON OXIDES)
Colorona® Sienna Fine, Merck®, CI 77491 (IRON OXIDES), MICA
Colorona® Sienna, Merck®, MICA, CI 77491 (IRON OXIDES)
Colorona® Precious Gold, Merck®, Mica, CI 77891 (Titanium dioxide), Silica, CI 77491(Iron oxides), Tin oxide
Colorona® Sun Gold Sparkle MP 29, Merck®, MICA, TITANIUM DIOXIDE, IRON OXIDES, MICA, CI 77891, CI 77491 (EU)
Colorona® Mica Black, Merck®, CI 77499 (Iron oxides), Mica, CI 77891 (Titanium dioxide)
Colorona® Bright Gold, Merck, Mica, CI 77891 (Titanium dioxide), CI 77491(Iron oxides)
Colorona® Blackstar Gold, Merck®, MICA, CI 77499 (IRON OXIDES)
Colorona® SynCopper, Merck®, Synthetic Fluorophlogopite (and) Iron Oxides
Colorona® SynBronze, Merck®, Synthetic Fluorphlogopite (and) Iron Oxides

Further particularly preferred pigments with the trade name Xirona® are, for example:
Xirona® Golden Sky, Merck®, Silica, CI 77891 (Titanium Dioxide), Tin Oxide
Xirona® Caribbean Blue, Merck®, Mica, CI 77891 (Titanium Dioxide), Silica, Tin Oxide
Xirona® Kiwi Rose, Merck®, Silica, CI 77891 (Titanium Dioxide), Tin Oxide
Xirona® Magic Mauve, Merck®, Silica, CI 77891 (Titanium Dioxide), Tin Oxide
Xirona® Le Rouge, Merck®, Iron Oxides (and) Silica

In addition, particularly preferred pigments with the trade name Unipure® are, for example:
Unipure® Red LC 381 EM, Sensient® CI 77491 (Iron Oxides), Silica
Unipure® Black LC 989 EM, Sensient®, CI 77499 (Iron Oxides), Silica
Unipure® Yellow LC 182 EM, Sensient®, CI 77492 (Iron Oxides), Silica

Also particularly preferred pigments with the trade name Flamenco® are, for example:
Flamenco® Summit Turquoise T30D, BASF®, Titanium Dioxide (and) Mica
Flamenco® Super Violet 530Z, BASF®, Mica (and) Titanium Dioxide

In a further embodiment, the agent (a) and/or agent (b) used in the process may also comprise one or more colorant compounds from the group of organic pigments.

The organic pigments are correspondingly insoluble organic dyes or colorants which may be selected, for example, from the group of nitroso, nitro-azo, xanthene, anthraquinone, isoindolinone, isoindoline, quinacridone, perinone, perylene, diketopyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, the process is exemplified in that the agent (a) and/or the agent (b) comprises at least one colorant compound from the group of organic pigments selected from the group of carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof.

The organic pigment can also be a color varnish. As contemplated herein, the term color varnish means particles comprising a layer of absorbed dyes, the unit of particle and dye being insoluble under the above mentioned conditions. The particles can, for example, be inorganic substrates, which can be aluminum, silica, calcium borosilate, calcium aluminum borosilicate or even aluminum.

For example, alizarin color varnish can be used.

In a further embodiment of the process, the agent (a) and/or the agent (b) may also contain one or more colorant compounds from the group of organic pigments.

In another particularly preferred embodiment, a process is exemplified in that the agent (a) and/or the agent (b) comprises at least one colorant compound from the group of organic pigments selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In particular, preference is given to a process in which agent (a) and/or agent (b) further comprises a colorant compound from the group of pigments and/or substantive dyes, the colorant compound comprising a pigment from the group of organic pigments.

Even more preferred is a process in which agent (a) and/or agent (b) further comprises a colorant compound from the group of pigments and/or direct dyes, the colorant compound comprising at least one pigment from the group of organic pigments and the organic pigment having a particle diameter in the range from about 100 to about 1000 nm, more preferably about 150 nm to about 700 nm.

Also suitable colorant compounds from the group of pigments are inorganic and/or organic pigments modified with a polymer. The polymer modification can, for example, increase the affinity of the pigments to the respective material of the at least one layer.

In agent (a) and/or agent (b) it is also possible to use so-called metal effect pigments as colorant.

In particular, the metal effect pigments may include pigments based on a lamellar substrate platelet, pigments based on lenticular substrate platelets, and/or pigments based on substrate platelets comprising “vacuum metallized pigments” (VMP). In these metal effect pigments, the substrate platelets comprise a metal, preferably aluminum, or an alloy. Metal substrate platelet-based metal effect pigments preferably have a coating which, among other things, acts as a protective layer.

Suitable metallic effect pigments include, for example, the pigments Alegrace® Marvelous, Alegrace® Gorgeous or Alegrace® Aurous from Schlenk® Metallic Pigments.

Also suitable metal effect pigments are the aluminum-based pigments of the SILVERDREAM® series and the pigments based on aluminum or on copper/zinc-containing metal alloys of the VISIONAIRE® series from Eckart®.

Due to their excellent light and temperature stability, the use of the above pigments in agent (a) and/or (b) is particularly preferred. It is also preferred if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D₅₀ of about 1 to about 50 µm, preferably about 5 to about 45 µm, preferably about 10 to about 40 µm, in particular about 14 to about 30 µm. The average particle size D₅₀, for example, can be determined using dynamic light scattering (DLS).

In a further preferred embodiment, the process is exemplified in that the agent (a) -based on the total weight of the agent (a) - further comprises one or more color-imparting compound(s) in the form of pigments in a total amount of from about 0.01 to about 10 wt.%, preferably from about 0.1 to about 8 wt.%, more preferably from about 0.2 to about 6 wt.% and most preferably from about 0.5 to about 4.5 wt.%.

In a further, likewise preferred embodiment, the process is exemplified in that the agent (b) - based on the total weight of the agent (b) - further comprises one or more color-imparting compound(s) in the form of pigments in a total amount of from about 0.01 to about 10 wt.%, preferably from about 0.1 to about 8 wt.%, more preferably from about 0.2 to about 6 wt.% and very particularly preferably from about 0.5 to about 4.5 wt.%.

As colorant compound(s), the agents (a) and/or agents (b) used in the process may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments. Preferably, the direct dyes within the meaning of the present disclosure have a solubility in water (760 mmHg) at 25° C. of more than 1 g/L.

Direct dyes can be divided into anionic, cationic and non-ionic direct dyes.

In a further preferred embodiment, the process is exemplified in that the agent (a) and/or the agent (b) further comprises as coloring compound at least one anionic, cationic and/or nonionic direct dye.

In a further preferred embodiment, the process is exemplified in that the agent (a) and/or the agent (b) further comprises at least one colorant compound selected from the group of anionic, nonionic, and/or cationic direct dyes.

Suitable cationic direct dyes include Basic Blue 7, Basic Blue 26, Basic Violet 2 and Basic Violet 14, Basic Yellow 57, Basic Red 76, Basic Blue 16, Basic Blue 347 (Cationic Blue 347 / Dystar), HC Blue No. 16, Basic Blue 99, Basic Brown 16, Basic Brown 17, Basic Yellow 57, Basic Yellow 87, Basic Orange 31, Basic Red 51 Basic Red 76

As non-ionic direct dyes, non-ionic nitro and quinone dyes and neutral azo dyes can be used. Suitable nonionic direct dyes are those known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino] -3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid, and 2-chloro-6-ethylamino-4-nitrophenol.

In the course of the work leading to the present disclosure, it has been found that dyeing of particularly high color intensity can be produced in particular with agents (a) and/or (b) containing at least one anionic direct dye.

In an explicitly quite particularly preferred embodiment, the process is therefore exemplified in that the agent (a) and/or the agent (b) further comprises at least one anionic direct dye as a coloring compound.

Anionic direct dyes are also called acid dyes. Acid dyes are direct dyes that have at least one carboxylic acid group (—COOH) and/or one sulfonic acid group (—SO₃H). Depending on the pH, the protonated forms (—COOH, —SO₃H) of the carboxylic or sulfonic acid moieties are in equilibrium with their deprotonated forms (—COO^{—}, —SO₃^{—} present). The proportion of protonated forms increases with decreasing pH. If direct dyes are used in the form of their salts, the carboxylic acid groups or sulfonic acid groups are present in deprotonated form and are neutralized with corresponding stoichiometric equivalents of cations to maintain electro neutrality. The acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1 g/L.

The alkaline earth salts (such as calcium salts and magnesium salts) or aluminum salts of acid dyes often have poorer solubility than the corresponding alkali salts. If the solubility of these salts is below 0.5 g/L (25° C., 760 mmHg), they do not fall under the definition of a direct dye.

A key feature of acid dyes is their ability to form anionic charges, with the carboxylic or sulfonic acid groups responsible for this usually attached to various chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In the context of one embodiment, a process for dyeing keratinous material is thus preferred, which is exemplified in that the agent (a) and/or the agent (b) further comprises at least one anionic direct dye as coloring compound, which is selected from the group of the nitrophenylenediamines, the nitroaminophenols, the azo dyes, the anthraquinone dyes, the triarylmethane dyes, the xanthene dyes, the rhodamine dyes, the oxazine dyes and/or the indophenol dyes, the dyes from the above-mentioned group each containing at least one carboxylic acid group (—COOH), a sodium carboxylate group (—COONa), a potassium carboxylate group (—COOK), a sulfonic acid group (-SO₃H), a sodium sulfonate group (—SO₃Na) and/or a potassium sulfonate group (—SO₃K).

For example, one or more compounds from the following group can be selected as particularly well suited acid dyes: Acid Yellow 1 (D&C Yellow 7, Citronin A, Ext. D&C Yellow No. 7, Japan Yellow 403,CI 10316, COLIPA n° B001), Acid Yellow 3 (COLIPA n° : C 54, D&C Yellow N° 10, Quinoline Yellow, E104, Food Yellow 13), Acid Yellow 9 (CI 13015), Acid Yellow 17 (CI 18965), Acid Yellow 23 (COLIPA n° C 29, Covacap Jaune W 1100 (LCW), Sicovit Tartrazine 85 E 102 (BASF®), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1;CI 20170;KATSU201;nosodiumsalt;Brown No.201;RESORCIN BROWN;ACID ORANGE 24;Japan Brown 201;D & C Brown No.1), Acid Red 14 (C.I.14720), Acid Red 18 (E124, Red 18; CI 16255), Acid Red 27 (E 123, CI 16185, C-Rot 46, Real red D, FD&C Red Nr.2, Food Red 9, Naphthol red S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI 27290), Acid Red 87 (Eosin, CI 45380), Acid Red 92 (COLIPA n° C53, CI 45410), Acid Red 95 (CI 45425, Erythtosine,Simacid Erythrosine Y), Acid Red 184 (CI 15685), Acid Red 195, Acid Violet 43 (Jarocol Violet 43, Ext. D&C Violet n° 2, C.I. 60730, COLIPA n° C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n° 401, Naphthalene Black 10B, Amido Black 10B, CI 20 470, COLIPA n° B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. An agitator is added. Then add 100 ml of water. This mixture is heated to 25° C. on a magnetic stirrer while stirring. It is stirred for 60 minutes. The aqueous mixture is then visually assessed. If there are still undissolved radicals, the amount of water is increased - for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1 g/L.

Acid Yellow 1 is called 8-hydroxy-5,7-dinitro-2-naphthalenesulfonic acid disodium salt and has a solubility in water of at least 40 g/L (25° C.).

Acid Yellow 3 is a mixture of the sodium salts of mono- and disulfonic acids of 2-(2-quinolyl)-1H-indene-1,3(2H)-dione and has a water solubility of 20 g/L (25° C.).

Acid Yellow 9 is the disodium salt of 8-hydroxy-5,7-dinitro-2-naphthalenesulfonic acid, its solubility in water is above 40 g/L (25° C.).

Acid Yellow 23 is the trisodium salt of4,5-dihydro-5-oxo-1-(4-sulfophenyl)-4-((4-sulfophenyl)azo)-1H-pyrazole-3-carboxylic acid and readily soluble in water at 25° C.

Acid Orange 7 is the sodium salt of 4-[(2-hydroxy-1-naphthyl)azo]benzene sulfonate. Its water solubility is more than 7 g/L (25° C.).

Acid Red 18 is the trisodium salt of 7-hydroxy-8-[(E)-(4-sulfonato-1-naphthyl)-diazenyl)]-1,3-naphthalenedisulfonate and has a very high water solubility of more than 20 wt. %.

Acid Red 33 is the disodium salt of 5-amino-4-hydroxy-3-(phenylazo)-naphthalene-2,7-disulphonate, its solubility in water is 2,5 g/L (25° C.).

Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).

Acid Blue 9 is the disodium salt of 2-({4-[N-ethyl(3-sulfonatobenzyl]amino]phenyl} {4-[(N-ethyl(3-sulfonatobenzyl)imino]-2,5-cyclohexadien-1-ylidene}methyl)-benzenesulfonate and has a solubility in water of more than 20 wt. % (25° C.).

A very particularly preferred process is therefore exemplified in that the agent (a) and/or the agent (b) further comprises at least one colorant compound selected from the group of anionic direct dyes, which is selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5,

Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The direct dye(s), in particular the anionic direct dyes, can be used in different amounts in the agent (a) and/or the agent (b) depending on the desired color intensity. Particularly good results were obtained when the agent (a) and/or the agent (b) - in each case based on its total weight - also contains one or more direct dyes as colorant compound in a total amount of from about 0.01 to about 10 wt.%, preferably from about 0.1 to about 8 wt.%, more preferably from about 0.2 to about 6 wt.% and very particularly preferably from about 0.5 to about 4.5 wt.%.

In a further preferred embodiment, the process is exemplified in that the agent (a) and/or the agent - based on the total weight of the agent (a) and/or the agent (b) - further comprises one or more direct dyes as colorant compound in a total amount of from about 0.01 to about 10 wt.%, preferably from about 0.1 to about 8 wt.%, more preferably from about 0.2 to about 6 wt.% and most preferably from about 0.5 to about 4.5 wt.%.

The following discloses preferred embodiments of the method with respect to the color-imparting compounds:
1. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore
      at least one coloring compound comprising at least one inorganic pigment selected from the group of colored metal oxides, metal hydroxides, metal oxide hydrates, silicates, metal sulfides, complex metal cyanides, metal sulfates, bronze pigments and mixtures thereof,
   application of an agent (b) to the keratinous material, wherein the agent (b) comprises:
      (b1) at least one sealing reagent.
2. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one coloring compound comprising at least one inorganic pigment selected from the group of colored metal oxides, metal hydroxides, metal oxide hydrates, silicates, metal sulfides, complex metal cyanides, metal sulfates, bronze pigments and mixtures thereof and at least one pigment selected from the group of pigments based on a lamellar metallic substrate platelet, pigments based on a lenticular metallic substrate platelet, pigments based on a metallic substrate platelet comprising a vacuum metallized pigment (VMP) and mixtures thereof,
   application of an agent (b) to the keratinous material, wherein the agent (b) comprises:
      (b1) at least one sealing reagent.
3. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one colorant compound comprising at least one inorganic pigment selected from the group of colored metal oxides, metal hydroxides, metal oxide hydrates, silicates, metal sulfides, complex metal cyanides, metal sulfates, bronze pigments, and mixtures thereof, and at least one pigment comprising α) a substrate platelet comprising mica, and β) a coating comprising at least a first metal oxide (hydrate) layer comprising TiO₂, SnO₂ and/or iron oxide(s),
   application of an agent (b) to the keratinous material, wherein the agent (b) comprises:
      (b1) at least one sealing reagent.
4. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one coloring compound comprising at least one inorganic pigment selected from the group of colored metal oxides, metal hydroxides, metal oxide hydrates, silicates, metal sulfides, complex metal cyanides, metal sulfates, bronze pigments and mixtures thereof,
   application of an agent (b) to the keratinous material, wherein the agent (b) comprises:
      (b1) at least one sealant reagent comprising a film-forming polymer, and
      (b2) at least one colorant compound comprising at least one pigment selected from the group of lamellar metallic substrate platelet-based pigments, lenticular metallic substrate platelet-based pigments, metallic substrate platelet-based pigments comprising a vacuum metallized pigment (VMP), and mixtures thereof.
5. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one coloring compound comprising at least one inorganic pigment selected from the group of colored metal oxides, metal hydroxides, metal oxide hydrates, silicates, metal sulfides, complex metal cyanides, metal sulfates, bronze pigments and mixtures thereof,
   application of an agent (b) to the keratinous material, wherein the agent (b) comprises:
      (b1) at least one sealant reagent comprising a film-forming polymer, and
      (b2) at least one colorant compound comprising a pigment comprising α) a substrate platelet comprising mica, and β) a coating comprising at least a first metal oxide (hydrate) layer comprising TiO₂, SnO₂ and/or iron oxide(s).
6. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one coloring compound comprising at least one inorganic pigment selected from the group of colored metal oxides, metal hydroxides, metal oxide hydrates, silicates, metal sulfides, complex metal cyanides, metal sulfates, bronze pigments and mixtures thereof,
   application of an agent (b) to the keratinous material, wherein the agent (b) comprises:
      (b1) at least one sealant reagent comprising a film-forming polymer, and
      (b2) at least one colorant compound comprising a pigment comprising α) a substrate platelet comprising borosilicate glass, and β) a coating comprising at least a first metal oxide (hydrate) layer comprising TiO₂, SnO₂, SiO₂, and/or iron oxide(s).
7. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one colorant compound comprising at least one organic pigment selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof,
   application of an agent (b) to the keratinous material, wherein the agent (b) comprises:
      (b1) at least one sealing reagent.
8. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one coloring compound comprising at least one organic pigment selected from the group of carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof, and at least one pigment selected from the group of pigments based on a lamellar, metallic substrate platelet-based pigments, lenticular metallic substrate platelet-based pigments, metallic substrate platelet-based pigments comprising a vacuum metallized pigment (VMP), and mixtures thereof,
   application of an agent (b) to the keratinous material, wherein the agent (b) comprises:
      (b1) at least one sealing reagent.
9. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one colorant compound comprising at least one organic pigment selected from the group of carmine, quinacridone, phthalocyanine, sorghum, blue pigments having the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof, and at least one pigment comprising α) a substrate platelet comprising mica, and β) a coating comprising at least a first metal oxide (hydrate) layer comprising TiO₂, SnO₂ and/or iron oxide(s),
   application of an agent (b) to the keratinous material, wherein the agent (b) comprises:
      (b1) at least one sealing reagent.
10. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one colorant compound comprising at least one organic pigment selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof,
   application of an agent (b) to the keratinous material, wherein the agent (b) comprises:
      (b1) at least one sealant reagent comprising a film-forming polymer, and
      (b2) at least one colorant compound comprising at least one pigment selected from the group of lamellar metallic substrate platelet-based pigments, lenticular metallic substrate platelet-based pigments, metallic substrate platelet-based pigments comprising a vacuum metallized pigment (VMP), and mixtures thereof.
11. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one colorant compound comprising at least one organic pigment selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof,
   application of an agent (b) to the keratinous material, wherein the agent (b) comprises:
      (b1) at least one sealant reagent comprising a film-forming polymer, and
      (b2) at least one colorant compound comprising a pigment comprising α) a substrate platelet comprising mica, and β) a coating comprising at least a first metal oxide (hydrate) layer comprising TiO₂, SnO₂ and/or iron oxide(s).
12. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one colorant compound comprising at least one organic pigment selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof,
   application of an agent (b) to the keratinous material, wherein the agent (b) comprises:
      (b1) at least one sealant reagent comprising a film-forming polymer, and
      (b2) at least one colorant compound comprising a pigment comprising α) a substrate platelet comprising borosilicate glass, and β) a coating comprising at least a first metal oxide (hydrate) layer comprising TiO₂, SnO₂, SiO₂, and/or iron oxide(s).
13. A process for dyeing keratinous material, in particular human hair, comprising the following steps:
   Application of an agent (a) to the keratinous material, wherein the agent (a) comprises:
      (a1) at least one organic silicon compound selected from the group of silanes having one, two or three silicon atoms,
      (a2) an anionic, branched polysaccharide with a repeating tetra saccharide unit,
      (a3) at least one polyol,
      (a4) not more than about 25 wt.% of water, based on the total weight of the agent (a), and furthermore at least one coloring compound comprising at least one inorganic pigment selected from the group of iron oxides having a particle diameter in the range of 100 to 1,000 nm,
   application of an agent (b) to the keratinous material, wherein the agent (b) comprises:
      (b1) at least one sealing reagent.

The agents (a) and/or (b) may additionally contain one or more surfactants. The term surfactants refer to surface-active substances. A distinction is made between anionic surfactants comprising a hydrophobic radical and a negatively charged hydrophilic head group, amphoteric surfactants, which carry both a negative and a compensating positive charge, cationic surfactants, which in addition to a hydrophobic radical have a positively charged hydrophilic group, and non-ionic surfactants, which have no charges but strong dipole moments and are strongly hydrated in aqueous solution.

Zwitterionic surfactants are those surface-active compounds which carry at least one quaternary ammonium group and at least one -COO⁽⁻⁾ - or -SO₃⁽⁻⁾ group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example, cocoacylaminopropyl dimethyl ammonium glycinate, and 2-alkyl-3-carboxymethyl-3-hydroxyethyl imidazolines each having about 8 to about 18 C atoms in the alkyl or acyl group, and cocoacylaminoethyl hydroxyethyl carboxymethyl glycinate. A preferred zwitterionic surfactant is the fatty acid amide derivative known under the INCI name cocamidopropyl betaine.

Ampholytic surfactants are surface-active compounds which, in addition to a C₈ —C₂₄ alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about about 8 to about 24 C atoms in the alkyl group. Typical examples of amphoteric or zwitterionic surfactants are alkyl betaines, alkylamidobetaines, amino propionates, aminoglycinates, imidazolinium betaines and sulfobetaines.

Particularly preferred ampholytic surfactants are N-cocosalkylaminopropionate, cocosacylaminoethylaminopropionate and C₁₂ — C₁₈ —acylsarcosine.

The agents (a) and/or (b) may also additionally contain at least one nonionic surfactant. Suitable nonionic surfactants have been shown to be alkyl polyglycosides as well as alkylene oxide addition products to fatty alcohols and fatty acids, each with about 2 to about 30 moles of ethylene oxide per mole of fatty alcohol or fatty acid. Preparations with good properties are also obtained if they contain as non-ionic surfactants fatty acid esters of ethoxylated glycerol reacted with at least about 2 mol ethylene oxide.

Furthermore, the agents (a) and/or (b) may additionally also contain at least one cationic surfactant. Cationic surfactants are surfactants, i.e. surface-active compounds, each with one or more positive charges. Cationic surfactants contain only positive charges. Usually these surfactants are composed of a hydrophobic part and a hydrophilic head group, the hydrophobic part usually includes a hydrocarbon structure (e.g. one or two linear or branched alkyl chains) and the positive charge(s) being located in the hydrophilic head group. Examples of cationic surfactants are
quaternary ammonium compounds which may carry one or two alkyl chains with a chain length of about 8 to 2about 8 carbon atoms as hydrophobic radicals,
quaternary phosphonium salts substituted by one or more alkyl chains having a chain length of about 8 to about 28 carbon atoms or
tertiary sulfonium salts.

Furthermore, the cationic charge can also be part of a heterocyclic ring (e.g. an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of about 0.1 to about 45 wt.%, preferably about 1 to about 30 wt.%and most preferably about 1 to about 15 wt.% - based on the total weight of the respective agent.

Furthermore, the agents (a) and/or (b) may also contain at least one anionic surfactant. Anionic surfactants are surface-active agents with exclusively anionic charges (neutralized by a corresponding counter cation). Examples of anionic surfactants are fatty acids, alkyl sulfates, alkyl ether sulfates and ether carboxylic acids with about 12 to about 20 C atoms in the alkyl group and up to about 16 glycol ether groups in the molecule.

The anionic surfactants are used in a total quantity of about 0.1 to about 45 wt.%, preferably about 1 to about 30 wt.% and most preferably about 1 to about 15 wt.% - based on the total weight of the respective agent.

Agent (a) and/or agent (b) may further comprise a matting agent. Suitable matting agents include, for example, (modified) starches, waxes, talc and/or (modified) silicic acids. The amount of matting agent is preferably between about 0.1 and about 10 wt. % based on the total amount of agent (a) or agent (b). Preferably, agent (a) contains a matting agent.

The agent (a) and/or the agent (b) may further comprise a thickening agent.

When using agents (a) and/or (b), they must not be too thin and drip off the keratin material. For this reason, it may be preferred that the agent (a) and/or (b) contains a thickening agent.

In the context of one embodiment, a process for dyeing keratinous material is thus preferred, which is exemplified in that the agent (a) and/or the agent (b) further comprises a thickening agent.

Suitable thickeners include, for example, chemically modified celluloses, such as propyl cellulose, methyl ethyl cellulose, carboxymethyl cellulose, hydroxyethyl cellulose, methyl hydroxyethyl cellulose, ethyl hydroxyethyl cellulose, hydroxypropyl cellulose, hydroxypropylmethylcellulose, carboxymethylhydroxyethylcellulose, sulfoethylcellulose, carboxymethylsulfoethylcellulose, hydroxypropylsulfoethylcellulose, hydroxyethylsulfoethylcellulose, methylethylhydroxyethylcellulose, methlylsulfoethylcellulose and/or ethylsulfoethylcellulose.

In a preferred embodiment, a method for dyeing keratinous material is exemplified in that the agent (a) and/or the agent (b) further comprises a thickening agent selected from the group of propylcellulose, methyl ethylcellulose, carboxymethylcellulose, hydroxyethylcellulose, methylhydroxyethylcellulose, ethylhydroxyethylcellulose, hydroxypropylcellulose, hydroxypropylmethylcellulose, carboxymethylhydroxyethylcellulose, sulfoethylcellulose, carboxymethylsulfoethylcellulose, hydroxypropylsulfoethylcellulose, hydroxyethylsulfoethylcellulose, methylethylhydroxyethylcellulose, methlylsulfoethylcellulose, ethylsulfoethylcellulose, and mixtures thereof.

Particularly suitable thickeners are selected from hydroxyethyl cellulose, hydroxypropyl cellulose, hydroxypropyl methyl cellulose and mixtures thereof.

In a particularly preferred embodiment, a method for dyeing keratinous material is exemplified in that the agent (a) and/or the agent (b) further comprises a thickening agent selected from the group of hydroxyethylcellulose, hydroxypropylcellulose, hydroxypropylmethylcellulose and mixtures thereof.

Other suitable thickeners include galactomannans. Preferred galactomannans include galactomannans having the INCI designation Cyamopsis tetragonoloba gum (Guar Gum), galactomannans having the INCI designation Ceratonia Siliqua (Carob) Gum (Locust Bean Gum), galactomannans having the INCI designation Cassia Gum, and galactomannans having the INCI designation Caesalpinia Spinosa Gum (Tara Gum).

Accordingly, a process for dyeing keratinous material is particularly preferred in which agent (a) and/or agent (b) further comprises at least one galactomannan which is selected from the group of galactomannans with the INCI designation Cyamopsis tetragonoloba gum (Guar Gum), galactomannans with the INCI designation Ceratonia Siliqua (Carob) Gum (Locust Bean Gum), galactomannans with the INCI designation Cassia Gum and galactomannans with the INCI designation Caesalpinia Spinosa Gum (Tara Gum). In a particularly preferred embodiment, the galactomannan comprises a galactomannan having the INCI designation Caesalpinia Spinosa Gum (Tara Gum).

The amount of thickener is preferably between 0.1 and 10 wt.%, in each case based on the total amount of agent (a) and/or agent (b).

The agents may also contain other active ingredients, auxiliaries and additives, such as solvents; fatty ingredients such as C₈-C₃₀ fatty acid triglycerides, C₈-C₃₀ fatty acid monoglycerides, C₈-C₃₀ fatty acid diglycerides and/or the hydrocarbons; structurants such as glucose, maleic acid and lactic acid, hair-conditioning compounds such as phospholipids, for example lecithin and cephalism; perfume oils, dimethyl isosorbide and cyclodextrins; fiber structure-improving active ingredients, in particular mono-, di- and oligosaccharides such as glucose, galactose, fructose, fructose and lactose; dyes for coloring the product; anti-dandruff active ingredients such as piroctone olamine, zinc omadine and climbazole; amino acids and oligopeptides; protein hydrolysates on an animal and/or vegetable basis, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; Polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; Fats and waxes such as fatty alcohols, beeswax, montan wax and kerosene; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate as well as PEG-3-distearate; and blowing agents such as propane-butane mixtures, N₂O, dimethyl ether, CO₂ and air.

The selection of these other substances will be made by the specialist according to the desired properties of the agents. With regard to other optional components and the quantities of these components used, explicit reference is made to the relevant manuals known to the specialist. The additional active ingredients and auxiliary substances are preferably used in the preparations as contemplated herein in quantities of about 0.0001 to about 25 wt.% each, in particular about 0.0005 to about 15 wt.%, based on the total weight of the respective agent.

### Process for Dyeing Keratinous Materials

In the procedure as contemplated herein, agents (a) and (b) are applied to the keratinous materials, in particular to human hair. Thus, agents (a) and (b) are the ready-to-use agents. The agents (a) and (b) are different.

In principle, agents (a) and (b) can be applied simultaneously or successively, whereby successive application is preferred.

The best results were obtained when agent (a) was first applied to the keratinous materials in a first step and agent (b) was applied in a second step.

Quite particularly preferred, therefore, is a process for treating keratinous material, in particular for coloring keratinous material, in particular human hair, comprising the following steps in the order indicated:
o in a first step, applying an agent (a) to the keratinous material, the agent comprising (a):
   (a1) at least one organic silicon compound selected from the group of silanes having one, two or three silicon atoms,
   (a2) an anionic, branched polysaccharide with a repeating tetra saccharide unit,
   (a3) at least one polyol and
   (a4) not more than 25 wt.% water, and
◯ In a second step, applying an agent (b) to the keratinous material, the agent comprising (b):
   (b1) at least one sealing reagent,
wherein at least one of the agents (a) and (b) further comprises at least one colorant compound selected from the group of pigments and/or direct dyes.

Moreover, in order to impart a high leaching resistance to the dyed keratinous material over a longer period of time, agents (a) and (b) are particularly preferably applied within one and the same dyeing process, which means that there is a period of a maximum of several hours between the application of agents (a) and (b).

In a further preferred embodiment, the method is exemplified in that agent (a) is applied first and agent (b) is applied thereafter, the period between the application of agents (a) and (b) being at most about 24 hours, preferably at most about 12 hours and particularly preferably at most about 6 hours.

A distinguishing feature of the agent (a) is its content of at least one reactive organic silicon compound (a1). The reactive organic silicon compound(s) (a1) undergoes an oligomerization or polymerization reaction and thus functionalizes the hair surface as soon as it comes into contact with it. In this way, a first, film is formed. In the second step of the process, a second agent (b) is now applied to the hair. During the application of the agent (b) comprising at least one film-forming polymer as sealing reagent (b1), the latter interacts with the silane film and is thus bound to the keratinous materials. During the application of agent (b) comprising at least one alkalizing agent or acidifying agent as sealing reagent (b1), the formation of the silane film is positively influenced. The desired coloring of the keratinous material is achieved by employing the coloring compound in agent (a) and/or in agent (b). The coloration can be achieved by a colored silane film (the colorant compound is only in agent (a)), by a colored polymer film (the coloring compound is only in agent (b) and this contains a film-forming polymer as sealing reagent (b1)) or by a colored silane film and by a colored polymer film (agents (a) and (b) each contain at least one coloring compound and agent (b) contains a film-forming polymer as sealing reagent (b1)).

In the context of a further embodiment, a method is very particularly preferred, comprising the following steps in the order indicated.
(1) Application of the agent (a) on the keratinous material,
(2) Allow the agent (a) to act for a period of about 10 seconds to about 10 minutes, preferably from about 10 seconds to about 5 minutes,
(3) if necessary, rinse the keratinous material with water,
(4) Application of agent (b) on the keratinous material,
(5) Allowing the agent (b) to act for a period of about 30 seconds to about 30 minutes, preferably from about 30 seconds to about 10 minutes,
(6) Rinse the keratinous material with water.

The rinsing of the keratinous material with water in steps (3) and (6) of the process is understood, as contemplated herein, to mean that only water is used for the rinsing process, without any other agents other than agents (a) and (b).

In step (1), agent (a) is first applied to the keratinous materials, in particular human hair.

After application, the agent (a) is left to act on the keratinous materials. In this context, application times from about 10 seconds to about 10 minutes, preferably from about 20 seconds to about 5 minutes and especially preferably from 30 seconds to 2 minutes on the hair have proven to be particularly beneficial.

In a preferred embodiment of the process, the agent (a) can now be rinsed from the keratinic materials before the agent (b) is applied to the hair in the subsequent step.

Dyeings with equally good wash fastness were obtained when agent (b) was applied to the keratinous materials that were still exposed to agent (a).

In step (4), agent (b) is now applied to the keratinous materials. After application, let the agent (b) act on the hair.

Even with a short contact time of the agent (b), the process allows the production of dyeing with particularly good intensity and wash fastness. Application times from about 10 seconds to about 10 minutes, preferably from about 20 seconds to about 5 minutes and most preferably from about 30 seconds to about 3 minutes on the hair have proven to be particularly beneficial.

In step (6), the agent (b) (and any agent (a) still present) is now rinsed out of the keratinous material with water.

In this embodiment, the sequence of steps (1) to (6) preferably takes place within about 24 hours.

Agent (a) contains, with the organic silicon compound(s), a class of highly reactive compounds that can undergo hydrolysis or oligomerization and/or polymerization when used. As a result of their high reactivity, these organic silicon compounds form a film on the keratinous material.

To avoid premature oligomerization or polymerization, it is of significant advantage to the user to prepare the ready-to-use agent (a) just prior to application.

In yet another embodiment, preferred is a method comprising the following steps in the order indicated.
(1) Preparation of an agent (a) having a water content of at most about 25 wt.%, based on the total weight of agent (a), by mixing a first agent (a′) and a second agent (a″), wherein
   ◯ the first agent (a′) comprises at least one organic silicon compound (a1) from the group of silanes having one, two or three silicon atoms, and
   the second agent (a″) comprises an anionic, branched polysaccharide having a repeating tetra saccharide unit (a2), at least one polyol (a3), water (a4) and at least one colorant compound selected from the group of pigments and/or direct dyes,
(2) Application of the agent (a) on the keratinous material,
(3) Allow the agent (a) to act for a period of about 10 seconds to about 10 minutes, preferably from about 10 seconds to about 5 minutes,
(4) if necessary, rinse the keratinous material with water,
(5) Application of agent (b) on the keratinous material,
(6) Allowing the agent (b) to act for a period of about 30 seconds to about 30 minutes, preferably from about 30 seconds to about 10 minutes,
(7) Rinse the keratinous material with water.

In order to be able to provide a formulation that is as stable as possible in storage, the agent (a′) itself is preferably formulated to be low in water or water-free.

In a preferred embodiment, a process is exemplified in that the agent (a′) - based on the total weight of the agent (a′) - contains a water content of from about 0.001 to about 10 wt.%, preferably from about 0.5 to about 9 wt.%, more preferably from about 1 to about 8 wt.% and very particularly preferably from about 1.5 to about 7 wt.%.

The agent (a″) may contain water. The water content of the agent (a″) is selected such that the content of water in the agent (a) formed by mixing the agents (a′) and (a″) is at most about 25 wt.%, based on the total weight of agent (a).

Within this embodiment, the ready-to-use agent (a) is prepared by mixing agents (a′) and (a″).

For example, the user may first stir or shake the agent (a′) containing the organic silicon compound(s) (a1) with the agent (a″). The user can now apply this mixture of (a′) and (a″) to the keratinous materials - either immediately after its preparation or after a short reaction time of about 10 seconds to about 30 minutes. Afterwards, the user can apply agent (b) as described above.

It may be preferred that the process further employs an agent (a″) comprising a colorant compound selected from the group of pigments and/or direct dyes comprising at least one pigment selected from the group of lamellar metallic substrate platelet-based pigments, lenticular metallic substrate platelet-based pigments, metallic substrate platelet-based pigments comprising a vacuum metallized pigment (VMP), and mixtures thereof.

In a preferred embodiment, a process is exemplified in that the agent (a‴) comprises at least one pigment selected from the group of pigments based on a lamellar metallic substrate platelet, pigments based on a lenticular metallic substrate platelet, pigments based on a metallic substrate platelet comprising a “vacuum metallized pigment” (VMP), and mixtures thereof.

In the context of a further embodiment, particularly preferred is a method comprising the following steps in the order indicated.
(1) Preparation of an agent (a) having a water content of at most 25 wt.%, based on the total weight of agent (a), by mixing a first agent (a′), a second agent (a″) and a third agent (a″), wherein
   ◯ the first agent (a′) comprises at least one organic silicon compound (a1) from the group of silanes having one, two or three silicon atoms,
   o the second agent (a″) comprises an anionic, branched polysaccharide having a repeating tetra saccharide unit (a2), at least one polyol (a3), water (a4) and at least one colorant compound selected from the group of pigments and/or direct dyes, and
   ◯ the third agent (a‴) comprises at least one pigment selected from the group of pigments based on a lamellar metallic substrate platelet, pigments based on a lenticular metallic substrate platelet, pigments based on a metallic substrate platelet comprising a “vacuum metallized pigment” (VMP), and mixtures thereof,
(2) Application of the agent (a) on the keratinous material,
(3) Allow the agent (a) to act for a period of about 10 seconds to about 10 minutes, preferably from about 10 seconds to about 5 minutes,
(4) if necessary, rinse the keratinous material with water,
(5) Application of agent (b) on the keratinous material,
(6) Allowing the agent (b) to act for a period of about 30 seconds to about 30 minutes, preferably from about 30 seconds to about 10 minutes,
(7) Rinse the keratinous material with water.

Within this embodiment, the ready-to-use agent (a) is prepared by mixing agents (a′), (a″) and (a‴).

Also in this embodiment, the water content of the agent (a″) is selected such that the content of water in the agent (a) formed by mixing the agents (a′), (a″) and (a″) is at most about 25 wt.%, based on the total weight of agent (a). The agent (a″) preferably confectioned low in water or more preferably free of water.

For example, the user may first mix or shake the agent (a″) with the metal pigment agent (a‴) and then mix or shake the resulting mixture with the agent (a′) containing the organic silicon compound(s) (a1). The user can now apply this mixture of (a′), (a″) and (a‴) to the keratinous materials - either immediately after its preparation or after a short reaction time of about 10 seconds to about 20 minutes. Afterwards, the user can apply agent (b) as described above.

### Multi-component Packaging Unit (Kit-of-Parts)

To increase user convenience, the user is preferably provided with all the necessary agents in the form of a multi-component packaging unit (kit-of-parts).

A second subject matter of the present disclosure is therefore a multi-component packaging unit (kit-of-parts) for coloring keratinic material, comprehensively packaged separately from one another
a first container comprising an agent (a′), wherein the agent comprises (a′):
   (a1) at least one organic silicon compound selected from the group of silanes having one, two or three silicon atoms, and
a second container comprising an agent (a″), wherein the agent comprises (a″):
   (a2) alginic acid and/or its salt, and
a third container containing an agent (b), wherein the agent contains (b):
   (b1) at least one sealing reagent,
where the components (a1), (a2) and (b1) have been disclosed in detail above, and at least one of the agents (a″) and (b) further comprises at least one colorant compound selected from the group of pigments and/or direct dyes.

The organic silicon compounds (a1) from the group of silanes with one, two or three silicon atoms contained in the agent (a′) of the kit correspond to the organic silicon compounds (a1) that were also used in the agent (a) of the previously described process.

The alginic acid and/or its salt (a2) contained in the agent (a″) of the kit corresponds to the alginic acid and/or its salts (a2) that were also used in the agent (a) of the process described previously.

The sealing reagent (b1) contained in the agent (b) of the kit is the same as the sealing reagent (b1) used in the agent (b) of the previously described method.

In the context of a further embodiment, a multi-component packaging unit (kit-of-parts) for coloring keratinic material is preferably packaged separately from one another
a first container comprising an agent (a′), wherein the agent comprises (a′):
   at least one organic silicon compound (a1) from the group of silanes having one, two or three silicon atoms, and
a second container comprising an agent (a″), the agent comprising (a″):
   (a2) an anionic, branched polysaccharide with a repeating tetra saccharide unit,
   (a3) at least one polyol (a3),
   (a4) water and a colorant compound selected from the group of pigments and/or direct dyes comprising an organic pigment, and water, and
a third container containing an agent (b), wherein the agent contains (b):
   (b1) at least one sealing reagent,
wherein the components (a1), (a2) and (b1) have been disclosed in detail above.

In the context of still another embodiment, preferred is a multi-component packaging unit (kit-of-parts) for dyeing keratinous material, comprising separately prepared
a first container comprising an agent (a′), wherein the agent comprises (a′):
   at least one organic silicon compound (a1) from the group of silanes having one, two or three silicon atoms, and
a second container comprising an agent (a″), the agent comprising (a″):
   (a2) an anionic, branched polysaccharide with a repeating tetra saccharide unit,
   (a3) at least one polyol (a3),
   (a4) water and at least one colorant compound selected from the group of pigments and/or direct dyes,
a third container containing an agent (b), wherein the agent contains (b):
   (b1) at least one sealing reagent comprising a film-forming polymer, and furthermore at least one colorant compound selected from the group of pigments and/or direct dyes,
wherein the components (a1), (a2) and (b1) have been disclosed in detail above.

In the context of still another embodiment, preferred is a multi-component packaging unit (kit-of-parts) for dyeing keratinous material, comprising separately prepared
a first container comprising an agent (a′), wherein the agent comprises (a′):
   at least one organic silicon compound (a1) from the group of silanes having one, two or three silicon atoms, and
a second container comprising an agent (a″), the agent comprising (a″):
   (a2) an anionic, branched polysaccharide with a repeating tetra saccharide unit,
   (a3) at least one polyol (a3),
   (a4) water and at least one colorant compound selected from the group of pigments and/or direct dyes comprising an organic pigment,
a third container comprising an agent (a″), said agent (a‴):
   at least one colorant compound selected from the group of pigments and/or direct dyes, comprising at least one pigment selected from the group of pigments based on a lamellar metallic substrate platelet, pigments based on a lenticular metallic substrate platelet, pigments based on a metallic substrate platelet comprising a vacuum metallized pigment (VMP), and mixtures thereof,
a fourth container containing agent (b), wherein the agent contains (b):
   (b1) at least one sealing reagent,
wherein the components (a1), (a2), (a3) and (b1) have been disclosed in detail above.

It may be advantageous, in particular if the agent (b) comprises at least one colorant compound from the group of pigments and/or direct dyes, to prepare the ready-to-use agent (b) by mixing two agents (b′) and (b″). Within this embodiment, the sealing reagent (b1) and the at least one colorant compound selected from the group of pigments and/or direct dyes are prepared separately.

Preferred in the context of this further embodiment is a multi-component packaging unit (kit-of-parts) for dyeing keratinous material, comprising separately prepared
a first container comprising an agent (a′), wherein the agent comprises (a′):
   at least one organic silicon compound (a1) from the group of silanes having one, two or three silicon atoms, and
a second container comprising an agent (a″), the agent comprising (a″):
   (a2) an anionic, branched polysaccharide with a repeating tetra saccharide unit,
   (a3) at least one polyol (a3),
   (a4) water and, if desired, at least one colorant compound selected from the group of pigments and/or direct dyes,
a third container comprising an agent (b′), wherein the agent comprises (b′):
   (b1) at least one sealant reagent comprising a film-forming polymer, and
a fourth container comprising an agent (b″), the agent comprising (b″):
   at least one colorant compound selected from the group of pigments and/or direct dyes,
wherein the components (a1), (a2) and (b1) have been disclosed in detail above.

In the above embodiments, it may be preferred that the agents (b′) and/or (b) further comprise a thickening agent.

A kit-of-parts is further preferred wherein the agent (b) comprises a thickening agent selected from the group of ethyl cellulose, hydroxypropyl cellulose, hydroxypropyl methyl cellulose, and mixtures thereof.

A multi-component kit-of-parts is also preferred, wherein the agent (a″) and the agent (b) each comprise a thickening agent selected from the group of ethyl cellulose, hydroxyethyl cellulose, hydroxypropyl cellulose, hydroxypropyl methyl cellulose, and mixtures thereof.

Concerning the further preferred embodiments of the multicomponent packaging unit, mutatis mutantis what has been said about the process applies.

Oligo and polymerization reactions of the organic silicon compound (a1) are already initiated when agents (a′) and (a″) are mixed or when agents (a′), (a″) and (a‴) are mixed.

It has proved to be a major challenge to optimally adjust the oligo and polymerization rate of the organic silicon compound (a1), i.e. the rate at which the silane film forms on the keratin material, to the application conditions.

When applied to human hair, for example, too fast an oligo and polymerization rate will result in polymerization being completed before all hair sections have been treated. Polymerization that is too fast makes whole-head treatment impossible. In the dyeing process, the excessively fast polymerization manifests itself in an uneven color result, so that the sections that were treated last are only poorly colored.

On the other hand, if polymerization is too slow, all areas of the keratin material can be treated without time pressure, but this increases the application time.

Surprisingly, it has been shown that the combination of anionic branched polysaccharide with a repeating tetra saccharide unit (a2) and at least one polyol (a3), as well as the limitation of the water content in the agent (a) to a maximum of about 25 wt.%, based on the total weight of the agent (a), not only leads to improved adhesion of the colorant compound to the keratinous material, but also to an optimum oligo and polymerization rate of the organic silicon compound (a1).

### EXAMPLES

### Example 1

The following formulations have been produced (unless otherwise indicated, all figures are in wt. %)

Agent (a′)

| | |
|---|---|
| Agent (a′) | wt.-% |
| (3-Aminopropyl)triethoxysilane (a1) | 24 |
| Methyltriethoxysilane (a1) | 72 |
| Water | ad 100 |

Agent (a″)

| Agent (a″) | wt.-% |
|---|---|
| Pigment mixture (CI 12490, CI 74160 and CI 11680) | 5 |
| Biosaccharide Gum-4 (INCI) (a2) | 0.5 |
| Glycerin (a3) | 31 |
| 1,2-propylene glycol (a3) | 28 |
| Ethanol | 10 |
| Perfume | 0.5 |
| Water (a4) | 25 |

The ready-to-use agent (a) was prepared by mixing 5 g of agent (a′) and 15 g of agent (a″). The pH value of the agent (a) was adjusted to a value of 10.5 by adding ammonia or lactic acid.

Agent (b)

| | |
|---|---|
| Agent (b) | wt.-% |
| Ethylene/Sodium Acrylate Copolymer (b1) (25% solution) | 15 |
| Hydroxyethyl cellulose | 2 |
| Water | ad 100 |

The agent (a) was massaged into one strand of hair at a time (Kerling, Euronatural hair white), and left to act for 1 minute. The agent (a) was then rinsed with water.

Subsequently, agent (b) was applied to the hair strand, left to act for 5 minute and then also rinsed with water.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for dyeing keratinous material, comprising the following steps:
applying an agent (a) to the keratinous material, wherein the agent (a) comprises:
(a1) an organic silicon compound (a1) selected from the group of silanes having one, two or three silicon atoms,
(a2) an anionic, branched polysaccharide (a2) with a repeating tetra saccharide unit,
(a3) a polyol (a3) and
(a4) not more than about 25 wt.% water, based on a total weight of the agent (a), and
applying an agent (b) to the keratinous material, wherein the agent (b) comprises:
(b1) a sealing reagent,
wherein at least one of the agents (a) and (b) further comprises a colorant compound selected from the group of pigments and/or direct dyes.

2. The method according to claim 1, wherein the agent (a) comprises the organic silicon compound (a1) of a formula (I) and/or (II) 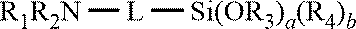
where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a stands for an integer from 1 to 3, and
b stands for the integer 3 - a, and
wherein in the organic silicon compound of formula (II) 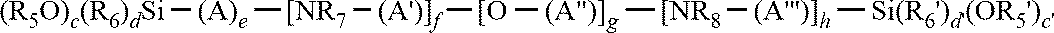
R5, R5′, R5″, R6, R6′ and R6″ independently represent a C₁-C₆ alkyl group,
A, A′, A″, A‴ and A⁗ independently represent a linear or branched divalent C₁-C₂₀ alkylene group,
R₇ and Rs independently represent a hydrogen atom, a C₁-C₆ alkyl group, a hydroxy C₁-C₆ alkyl group, a C₂-C₆ alkenyl group, an amino C₁-C₆ alkyl group or a group of formula (III) 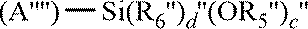
wherein
c stands for an integer from 1 to 3,
d stands for the integer 3 - c,
c′ stands for an integer from 1 to 3,
d′ stands for the integer 3 - c′,
c″ stands for an integer from 1 to 3,
d″ stands for the integer 3 - c″,
e stands for 0 or 1,
f stands for 0 or 1,
g stands for 0 or 1,
h stands for 0 or 1,
provided that at least one of the radicals e, f, g and h is different from 0.

3. The method according to claim 1, wherein the agent (a) comprises the organic silicon compound (a1) of formula (I), 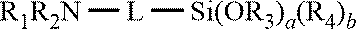
where
R₁, R₂ both represent a hydrogen atom,
L represents a linear, bivalent C₁-C₆-alkylene group,
R₃, R₄ independently represent a methyl group or an ethyl group,
a stands for the number 3 and
b stands for the number 0.

4. The method according to claim 2, wherein the organic silicon compound (a1) of formula (I) is selected from the group of:
(3-Aminopropyl)triethoxysilane
(3-Aminopropyl)trimethoxysilane
1-(3-Aminopropyl)silantriol
(2-Aminoethyl)triethoxysilane
(2-Aminoethyl)trimethoxysilane
1-(2-Aminoethyl)silantriol
(3-Dimethylaminopropyl)triethoxysilane
(3-Dimethylaminopropyl)trimethoxysilane
1-(3-Dimethylaminopropyl)silantriol
(2-Dimethylaminoethyl)triethoxysilane.
(2-dimethylaminoethyl)trimethoxysilane and/or
1-(2-Dimethylaminoethyl)silantriol.

5. The method according to claim 2, wherein the agent (a) comprises the organic silicon compound (a1) of the formula (II), 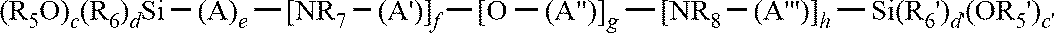
where
e and f both stand for the number 1,
g and h both stand for the number 0,
A and A′ independently represent a linear, bivalent C₁-C₆ alkylene and
R7 represents a hydrogen atom, a methyl group, a 2-hydroxyethyl group, a 2-alkenyl group, a 2-aminoethyl group or a group of the formula (III).

6. The method according to claim 1, wherein the agent (a) comprises the organic silicon compound (a1) of formula (IV), 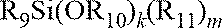
where
R₉ stands for a C₁-C₁₈ alkyl group,
R₁₀ represents a hydrogen atom or a C₁-C₆ alkyl group,
R₁₁ represents a C₁-C₆ alkyl group
k is an integer from 1 to 3, and
m stands for the integer 3 - k.

7. The method according to claim 6, wherein the agent (a) comprises the organic silicon compound (a1) of formula (IV) selected from the group of:
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Octadecyltrimethoxysilane,
Octadecyltriethoxysilane and
Mixtures of these.

8. The method according to claim 1, wherein the organic silicon compound (a1) comprises at least two structurally different compounds.

9. The method according to claim 1, wherein the sealing reagent comprises a compound selected from the group of: film-forming polymers, alkalizing agents, acidifying agents, and mixtures thereof.

10. The method according to claim 1, wherein the polysaccharide (a2) comprises D-glucose, L-fucose, and D-glucuronic acid in a ratio of about 2:1:1.

11. The method according to claim 1, wherein the agent (a) further comprises at least one colorant compound selected from the group of pigments and/or direct dyes.

12. The method according to claim 1, wherein the polyol (a3) comprises 2-propylene glycol and/or glycerol.

13. The method according to claim 1, wherein the colorant compound comprises an organic pigment selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof.

14. The method according to claim 1, wherein the colorant compound comprises an organic pigment selected from the group of carmine, quinacridone, phthalocyanine, sorghum, blue pigments having the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof and wherein the colorant compound further comprises at least one pigment selected from the group of pigments based on a lamellar, lamellar metallic substrate platelet-based pigments, lenticular metallic substrate platelet-based pigments, metallic substrate platelet-based pigments comprising vacuum metallized pigment (VMP) and mixtures thereof and/or at least one pigment based on natural or synthetic mica coated with at least one metal oxide and/or a metal oxychloride.

15. A kit-of-parts for dyeing keratinous material, comprising separately packaged
a first container containing an agent (a′), wherein the agent (a′) comprises : (a1) an organic silicon compound (a1) selected from the group of silanes having one, two or three silicon atoms, and
a second container comprising containing an agent (a″), the agent (a″) comprising: (a2) an anionic, branched polysaccharide (a2) with a repeating tetra saccharide unit,
(a3) a polyol (a3) and (a4) not more than about 25 wt.% water, based on a total weight of the sum of agent (a′) and agent (a″), and
- a third container containing an agent (b), wherein the agent (b₎ comprises: (b1) a sealing reagent, wherein at least one of the agents (a″) and (b) further comprises a colorant compound selected from the group of pigments and/or direct dyes.

16. The method of claim 3, wherein L represents a propylene group (—CH₂—CH₂—CH₂—) or an ethylene group (—CH₂—CH₂—).

17. The kit-of-parts of claim 15, wherein the organic silicon compound (a1) comprises a compound of a formula (I) and/or a formula (II) 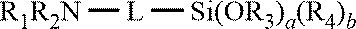
where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a stands for an integer from 1 to 3, and
b stands for the integer 3 - a, and
wherein in the organic silicon compound of formula (II) 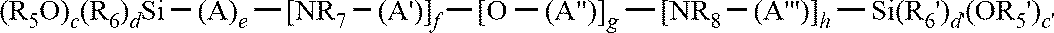
R5, R5′, R5″, R6, R6′ and R6″ independently represent a C₁-C₆ alkyl group,
A, A′, A″, A‴ and A⁗ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III) 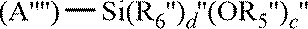
wherein
c stands for an integer from 1 to 3,
d stands for the integer 3 - c,
c′ stands for an integer from 1 to 3,
d′ stands for the integer 3 - c′,
c″ stands for an integer from 1 to 3,
d″ stands for the integer 3 - c″,
e stands for 0 or 1,
f stands for 0 or 1,
g stands for 0 or 1,
h stands for 0 or 1,
provided that at least one of the radicals e, f, g and h is different from 0.

18. The kit-of-parts of claim 15, wherein the agent (a) comprises the organic silicon compound (a1) of formula (IV), 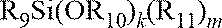
where
R₉ stands for a C₁-C₁₈ alkyl group,
R₁₀ represents a hydrogen atom or a C₁-C₆ alkyl group,
R₁₁ represents a C₁-C₆ alkyl group
- k is an integer from 1 to 3, and
- m stands for the integer 3 - k.

19. The kit-of-parts of claim 15, wherein the organic silicon compound (a1) comprises at least two structurally different compounds.

20. The kit-of-parts of claim 15, wherein the polysaccharide (a2) comprises D-glucose, L-fucose, and D-glucuronic acid in a ratio of about 2:1:1.
